(19) United States
(12) Patent Application Publication
(10) Pub. No.: US 2026/0258036 A1
(43) Pub. Date: Sep. 3, 2026

(54) INHIBITORS OF HUMAN ALDOSTERONE SYNTHASE (CYP11B2)

(71) Applicant: «TARGET MEDICALS» LIMITED LIABILITY COMPANY, Moscow (RU)

(72) Inventors: Andrei Aleksandrovich GILEP, Minsk (BY); Natallia Vladimirovna STRUSHKEVICH, Minsk (BY); Pascal GEORGE, Longvilliers (FR)

(21) Appl. No.: 19/110,329

(22) PCT Filed: Sep. 18, 2023
(86) PCT No.: PCT/RU2023/050224

§ 371 Date: Mar. 10, 2025

(30) Foreign Application Priority Data
Nov. 10, 2022 (RU) 2022129211
Apr. 10, 2023 (RU) 2023108992
Aug. 7, 2023 (RU) 2023120694

(51) Int. Cl. C07D 471/04; A61K 31/444; A61K 31/4545; A61K 31/496; A61K 31/497; A61K 31/4995; A61K 31/501; A61K 31/506; A61K 31/5377; A61K 31/541; A61K 31/551; C07D 519/00

(52) U.S. Cl. CPC C07D 471/04; A61K 31/444; A61K 31/4545; A61K 31/496; A61K 31/497; A61K 31/4995; A61K 31/501; A61K 31/506; A61K 31/5377; A61K 31/541; A61K 31/551; C07D 519/00

(57) ABSTRACT

This invention relates to organic chemistry, pharmacology, medicine, namely the invention relates to compounds for use in inhibiting the activity of human cytochrome 11B2 (CYP11B2) and their use in the treatment and/or prevention of various diseases and disorders that are mediated or supported by the activity of the hormone aldosterone. The compounds are characterized by the general formula (I), wherein R₁, R₂, R₃, R₄ and R₅ have the meanings specified in the description. The present invention also relates to pharmaceutical compositions containing the compounds of the invention and methods for preparing these compounds.

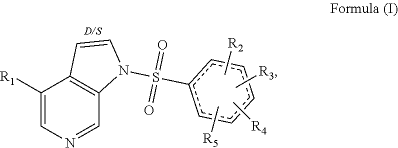

### TECHNICAL FIELD

This invention relates to organic chemistry, pharmacology, medicine, namely the invention relates to compounds for use in inhibiting the activity of human cytochrome 11 B2 (CYP11B2) and their use in the treatment and/or prevention of various diseases and disorders that are mediated or supported by the activity of the hormone aldosterone. The present invention also relates to pharmaceutical compositions containing the compounds of the invention and methods for preparing these compounds.

### PRIOR ART

Aldosterone is a steroid hormone whose main physiological role is to maintain sodium and potassium balance by regulating cation exchange (Na+ reabsorption and K+ secretion) in the distal nephron, which in turn leads to an increase in blood pressure. In addition to its physiological role, aldosterone is also involved in the pathogenesis of diseases or pathological conditions caused (primary hyperaldosteronism) and/or accompanied by its hypersynthesis (secondary hyperaldosteronism), including a number of diseases of the cardiovascular system, kidneys and metabolic syndrome.

Modern approaches to the treatment of conditions caused and/or accompanied by hypersynthesis of aldosterone include the impact on various levels of the renin-angiotensin-aldosterone system (RAAS). Modern standards of medical care are represented by such classes of drugs as renin inhibitors (IRs), angiotensin II receptor blockers (ARBs), angiotensin-converting enzyme inhibitors (ACEIs), and mineralocorticoid receptor (MR) blockers. However, in 25-50% of patients treated with IRs, ARBs, and ACE inhibitors alone or in combination, serum aldosterone levels eventually return to pretreatment levels or higher (aldosterone breakthrough) (Bomback et al., 2007, Nature Clinical Practice. Nephrology, 3, 486-492; Bomback et al., 2012, J Am Soc Hypertens, 6(5):338-45), and the introduction of MR blockers leads to a compensatory increase in serum aldosterone levels (Pitt at al., 2013, Eur Heart J, 34:2453-2463; Calhoun et al., 2011, Circulation, 124, 1945-1955). In this regard, to date, there is still no solution that can fully cover all the pathological effects of aldosterone in humans, including both MR-mediated and non-MR-mediated effects. The latter include the following: a) acting through GPCR receptors, aldosterone has a so-called “rapid effect”, leading to apoptosis and preventing the proliferation of vascular endothelial and smooth muscle cells [Namsolleck et al., 2014, Nephrology, Dialysis, Transplantation, 29 (Suppl. 1), i62-i68), impaired vasoconstriction (Feldman et al., 2013, Clinical and Experimental Pharmacology & Physiology, 40, 916-921), and endothelial inflammation [Ziwei Tang et al., 2021, International Journal of Endocrinology, Volume 2021, Article ID 5575927]. In addition, recent in vitro and ex vivo studies have shown that aldosterone, also through GPCR activation, enhances CYP11 B2 gene expression and therefore enhances its own biosynthesis and release (Caroccia et al., 2019, The Journal of Clinical Endocrinology & Metabolism, Volume 104, Issue 12, 6316-6324); b) damage to pancreatic beta cells (Luther et al., 2011, Diabetologia, 54, 2152-2163; Fang Chen et al., 2015, Scientific Reports volume 5, Article number: 13215 (2015), which can lead to tolerance to glucose c) interaction with IGF-I receptors in vascular smooth muscle cells with stimulation of elastin production by fibroblasts (Mitts et al., 2010, Journal of Investigative Dermatology, Volume 130, Issue 10, 2396-2406), to vascular remodeling and indicate a mechanism by which aldosterone may accelerate the development of atherosclerosis (Cascella et al., 2010, Endocrinology, 151(12):5851-64); d) induction of vasoconstriction through protein kinase C-dependent pathways, possibly through membrane receptors, which leads to deterioration of contractile and metabolic functions of the heart in ischemic hearts, therefore, an increase in plasma or cardiac tissue aldosterone levels may have an adverse effect in coronary heart disease (Fujita et al., 2005, Hypertension, 46(1):113-7); e) MR-independent activation of potassium current (IK1) by aldosterone has been shown to be the main mechanism explaining the increase in ventricular arrhythmia and sudden cardiac death accompanied by high aldosterone levels during ST-segment elevation myocardial infarction (STEMI) (Alexandre et al., 2015, PLoS One v.10(7); f) dimerization of the angiotensin II receptor (AT1) by aldosterone may play an important role in pathological conditions, including hypertension and atherosclerosis (Sinphitukkul et al., 2019, Arch Med Sci, 15(6):1589-1598). In addition, aldosterone has demonstrated a non-genomic endothelium-independent vasoconstrictor effect, also mediated through the AT1 receptor (Yamada et al., 2008, Cardiovasc Res, 79(1):169-78).

Based on the foregoing, it follows that the inhibition of aldosterone synthesis will have a therapeutic advantage over existing therapies, as well as overcome the limitations of existing standards of care.

Aldosterone is synthesized by a single enzyme, CYP11 B2 (aldosterone synthase), which catalyzes a 3-step conversion of 11-deoxycorticosterone (11-DOC) to aldosterone via corticosterone and 18-hydroxycorticosterone, so CYP11 B2 is a potential therapeutic target for inhibition of aldosterone synthesis, which may have an excellent pharmacodynamic profile. from that observed in other RAAS modulators.

From the foregoing, it clearly follows that agents capable of inhibiting CYP11 B2 (aldosterone synthase) can be used to treat and/or prevent a disease or condition that is mediated or maintained by hypersynthesis of aldosterone. Translation of data from in vitro studies on aldosterone synthase inhibition to in vivo results in both rodents and primates has been demonstrated for at least 7 compounds with different chemical structures, and moreover, 3 of them have shown an aldosterone-lowering effect in clinical trials: LC1699 (Novartis), R06836191 (Roche) and LY3045697 (Ely Lilly) [Aldosterone synthase inhibitors for cardiovascular diseases: A comprehensive review of preclinical, clinical and in silico data. Lenzini L et. al. Pharmacol Res. 2021; 163:105332. doi:10.1016/j.phrs.2020.105332; PMID: 33271294], [Inhibitors of Aldosterone Synthase. Weldon S M, Brown N F. Vitam Horm. 2019; 109:211-239. doi: 10.1016/bs.vh.2018.10.002; PMID: 30678857; clinical trials: NCT00817635, NCT01995383, NCT01821703]. Therefore, inhibition of aldosterone synthase activity under in vitro conditions will also be observed in vivo.

Moreover, it has been shown that a CYP11 B2-specific molecule can be used as a diagnostic marker for various diseases associated with CYP11 B2 overexpression. [Development of [18F]AldoView as the First Highly Selective Aldosterone Synthase PET Tracer for Imaging of Primary Hyperaldosteronism. Sander K et al. J Med Chem. 2021; 8; 64(13):9321-9329. doi: 10.1021/acs.jmedchem.1c00539. PMID: 34137616].

### DISCLOSURE OF THE INVENTION

### Brief Description of the Invention

In its multiple embodiments, the present invention provides novel chemicals capable of inhibiting the activity of the human CYP11 B2 enzyme, and their biological activity has been investigated. Thus, the invention relates to compounds, pharmaceutical compositions containing these compounds, the use of the compounds, methods of their use in the treatment of various diseases and disorders, as well as methods for obtaining these compounds.

### DETAILED DESCRIPTION OF THE INVENTION

The compounds disclosed herein inhibit the activity of the human CYP11 B2 enzyme and their biological activity has been investigated.

Thus, the invention relates to compounds, pharmaceutical compositions containing these compounds, the use of these compounds, methods of their use in the treatment of various diseases and disorders, as well as methods for obtaining these compounds.

Disclosed herein is a compound of formula (I) or pharmaceutically acceptable salts thereof, as defined herein after.

Disclosed herein is a compound of formula (I) or pharmaceutically acceptable salts thereof, as defined herein after, for use as a medicament.

Further disclosed herein is a compound of formula (I) as defined herein after, for use in lowering aldosterone levels in a subject.

Further disclosed herein is a compound of formula (I) as defined herein after, for use in the treatment and/or prevention of a disease or condition that is mediated or maintained by aldosterone hypersynthesis.

Further disclosed herein are compounds of formula (I) as defined herein after for use in the treatment of a disease selected in the group consisting in primary hyperaldosteronism (Conn's syndrome), chronic heart failure, chronic heart failure with preserved ejection fraction, left ventricular dysfunction, left ventricular hypertrophy, arterial hypertension, resistant arterial hypertension, pulmonary arterial hypertension, coronary heart disease, liver cirrhosis, metabolic syndrome, chronic kidney disease, glomerulosclerosis, glomerulonephritis, nephritic syndrome, focal segmental glomerulosclerosis, diabetic nephropathy, heart and vascular remodeling, postinfarction cardiosclerosis, atherosclerosis, increased collagen formation, endothelial dysfunction, hypokalemia, and insulin resistance.

Further disclosed herein are compounds of the Formula (I):

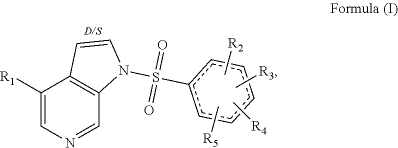

or a pharmaceutically acceptable salt, solvate or hydrate thereof, wherein:
R₁ is a 5-6 membered heteroaryl containing 0-1 oxygen, 0-1 sulfur and/or 0-4 nitrogen, optionally substituted with 1-5 substituents R1a,
R1a is independently selected and is hydrogen, deuterium, halogen, (C1-C3) alkyl, (C3-C5) cycloalkyl, —C(═O)—CH₃, partially or fully halogenated (C1-C3) alkyl, (C1-C3) alkylsulfonyl, cyano, N,N-dimethylamine, N-methylamine, —C(═O)—NH—(C1-C5)-alkyl;
R₂, R₃, R₄, R₅ are each independently selected and are:
   (i) hydrogen, deuterium, halogen, (C1-C3)-alkyl, (C3-C5)-cycloalkyl, —C(═O)—CH₃, partially or fully halogenated (C1-C3)alkyl, or
   (ii) —OH, —SH, or
   (iii) —(C1-C3)-alkoxy, 3-7-membered heterocyclyl containing 0-2 nitrogen atoms, 0-1 oxygen atom and/or 0-1 sulfur atom, (C3-C7)-cycloalkyl, —S—(C1-C3)-alkyl, —(C3-C7)-cycloalkyl-S—(C1-C3)-alkyl, wherein each of the above groups is optionally partially or completely substituted with substituents selected from —OH, deuterium, 1-4 halogen atoms, (C1-C3)-alkoxy, (C3-C7)-cycloalkyl, oxo group, —(C1-C3)-alkyl-(C3-C6)-cycloalkyl, (C1-C5)-alkyl, partially or fully halogenated (C1-C5) alkyl, (C2-C3) alkenyl, (C2-C3) alkynyl, (C1-C3) alkoxy, —NH₂, 3-7-membered heterocycle containing one oxygen atom, (C1-C3)-alkylamino, di(C1-C3)-alkylamino or 3-7-membered heterocycle containing one nitrogen atom and optionally 1-2 oxygen and/or sulfur heteroatoms; or
   (iv) 4-7-membered heterocyclyl containing 1-2 nitrogen atoms and 0-1 oxygen atom and/or 0-1 sulfur atom, necessarily substituted with 1-4 substituents selected from oxo group or —(C1-C3)-alkyl-(C3-C6)-cycloalkyl, and also optionally substituted with 1-3 substituents selected from deuterium, halogen, (C1-C5)-alkyl, partially or fully halogenated (C1-C5) alkyl, (C2-C3) alkenyl, (C2-C3) alkynyl and (C1-C3) alkoxy, or
   (v) —NRaRb where each Ra and Rb:
   is independently hydrogen, deuterium, (C3-C7)-cycloalkyl, —(C1-C3)-alkyl-N(CH₃)₂, (C1-C3)-alkyl, partially or fully halogenated (C1-C3)-alkyl, 3-7 membered heterocyclyl containing 0-1 oxygen atom, 0-1 sulfur atom and/or 0-4 nitrogen atoms, optionally substituted with 1-2 substituents selected from (C1-C3)-alkyl or partially or fully halogenated (C1-C3)-alkyl, 5-7-membered heteroaryl containing 0-1 oxygen atom, 0-1 sulfur atom and/or 0-4 nitrogen atoms, said heteroaryl being optionally substituted with 1-2 substituents selected from (C1-C3)-alkyl or partially or fully halogenated (C1-C3)-alkyl,
alternatively, Ra and Rb together with the nitrogen atom to which they are attached form a 4-7 membered heterocyclyl containing 1-2 nitrogen atoms and 0-1 oxygen atom and/or 0-1 sulfur atom, optionally substituted with 1-4 substituents selected from deuterium, halogen, (C1-C5)-alkyl, partially or fully halogenated (C1-C5)-alkyl, oxo group, —(C1-C3)-alkyl-(C3-C6)-cycloalkyl, (C2-C3)-alkenyl, (C2-C3)-alkynyl, (C1-C3) alkoxy or
alternatively, Ra and Rb together with the nitrogen atom to which they are attached form a 6-14-membered bicyclic heterocyclyl containing 1-2 nitrogen atoms and 0-1 oxygen and/or 0-1 sulfur atoms, optionally substituted with 1-4 substituents selected from deuterium, halogen, (C1-C5)-alkyl, partially or fully halogenated (C1-C5)-alkyl, (C2-C3)-alkenyl, (C2-C3) alkynyl, oxo group or —(C1-C3)-alkyl-(C3-C6)-cycloalkyl or (C1-C3) alkoxy;
D/S—means the presence of a double or single bond;
dashed bonds mean the rings are aromatic.

In another embodiment, the present invention includes compounds of formula (I) as defined above, at least two of R₂, R₃, R₄ and R₅ are other than hydrogen.

In another embodiment, the present invention includes compounds of formula (I) as defined above, at least three of R₂, R₃, R₄ and R₅ are other than hydrogen.

In another embodiment, the present invention includes compounds of formula (I) as defined above, R₂, R₃, R₄ and R₅ are other than hydrogen.

In another embodiment, the present invention includes compounds of formula (I) as defined above, R₂, R₃, R₄ and R₅ each is chosen independently and represent:
(i) hydrogen, deuterium, halogen, (C1-C3)-alkyl, (C3-C5)-cycloalkyl, —C(═O)—CH₃, partially or fully halogenated (C1-C3)-alkyl;
(ii) a group selected from (C1-C3)-alkoxy, (C3-C7)cycloalkyl, halogenated (C1-C3)alkoxy group containing 1-4 halogen atoms;
(iii) —NRaRb, where each Ra and Rb:
is independently hydrogen, deuterium, (C3-C7)-cycloalkyl, —(C1-C3)-alkyl-N(CH₃)₂, (C1-C3)-alkyl, partially or fully halogenated (C1-C3)-alkyl, 3-7 membered heterocyclyl containing 0-1 oxygen atom, 0-1 sulfur atom and/or 0-4 nitrogen atoms, optionally substituted with 1-2 substituents selected from (C1-C3)-alkyl, partially or fully halogenated (C1-C3)-alkyl, 5-7 membered heteroaryl containing 0-1 oxygen atom, 0-1 sulfur atom and/or 0-4 nitrogen atoms, optionally substituted with 1-2 substituents selected from (C1-C3)-alkyl, partially or fully halogenated (C1-C3)-alkyl,
alternatively, Ra and Rb together with the nitrogen atom to which they are attached form a 4-7 membered heterocyclyl containing 1-2 nitrogen atoms and 0-1 oxygen atom and/or 0-1 sulfur atom, optionally substituted with 1-2 substituents selected from (C1-C3)-alkyl, partially or fully halogenated (C1-C3)-alkyl, or
alternatively, Ra and Rb together with the nitrogen atom to which they are attached form a 6-14-membered bicyclic heterocyclyl containing 1-2 nitrogen atoms and 0-1 oxygen and/or 0-1 sulfur atom, optionally substituted with 1-4 substituents selected from deuterium, halogen, (C1-C3)-alkyl, partially or fully halogenated (C1-C3)-alkyl, (C2-C3)-alkenyl, (C2-C3)-alkynyl or (C1-C3)-alkoxy.

In another embodiment, in the compounds of formula (I) as defined above, at least one of R2, R3, R4 and R5 is at para position and each is independently selected and is: —OH, —SH, (C1-C3)-alkoxy, 3-7-membered heterocyclyl containing 0-2 nitrogen atom, 0-1 oxygen atom and/or 0-1 sulfur atom, (C3-C7)-cycloalkyl, —S—(C1-C3)-alkyl, (C3-C7)-cycloalkyl-S—(C1-C3)-alkyl, wherein each of the above groups is optionally partially or completely substituted with substituents selected from —OH, 1-4 halogen atoms, (C1-C3)-alkoxy, (C3-C7)-cycloalkyl, 3-7-membered heterocycle containing one oxygen atom, —NH₂, (C1-C3)-alkylamino, di(C1-C3)-alkylamino or 3-7-membered heterocycle containing one nitrogen atom and optionally 1-2 oxygen and sulfur heteroatoms; or
NRaRb, where each Ra and Rb:
is independently hydrogen, (C3-C7)-cycloalkyl, —(C1-C3)-alkyl-N(CH₃)₂, (C1-C3)-alkyl, partially or fully halogenated (C1-C3)-alkyl, 3-7 membered heterocycle containing 0-1 oxygen atom, 0-1 sulfur and 1-4 nitrogen atoms, optionally substituted with 1-2 substituents selected from (C1-C3)-alkyl or partially or fully halogenated (C1-C3)-alkyl, 5-7 membered heteroaryl containing 1-2 nitrogen atom, wherein said heteroaryl is optionally substituted with 1-2 substituents selected from (C1-C3)-alkyl or partially or fully halogenated (C1-C3)-alkyl,
alternatively, Ra and Rb together with the nitrogen atom to which they are attached form a 4-7 membered heterocyclyl containing 1-2 nitrogen atoms and 0-1 oxygen atom and/or 0-1 sulfur atom, optionally substituted with 1-2 substituents selected from (C1-C3)-alkyl, partially or fully halogenated (C1-C3)-alkyl, or
alternatively, Ra and Rb, together with the nitrogen atom to which they are attached, form a 6-14-membered bicyclic heterocyclyl containing 1-2 nitrogen atoms and 0-1 oxygen and/or 0-1 sulfur atoms, optionally substituted with 1-4 substituents selected from halogen, (C1-C3)-alkyl, partially or fully halogenated (C1-C3)-alkyl, (C2-C3)-alkenyl, (C2-C3) alkynyl or (C1-C3) alkoxy.

In another embodiment, in compounds of formula (I) as defined above, wherein R₁ is independently selected from pyridinyl, pyrazinyl, pyrimidinyl, pyridazinyl, oxazyl, R₁ is optionally substituted with a group selected from methyl, methylsulfonyl, halogen, methylamino, cyano and cyclopropyl.

In another embodiment, in compounds of formula (I) as defined above, wherein two of R₂, R₃, R₄, R₅ are in meta position and are a group selected from methyl or halogen, one of R₂, R₃, R₄, R₅ is hydrogen and one of R₂, R₃, R₄, R₅ is in para position and is a group selected from methoxy, isopropyloxy, cyclopropylmethoxy, difluoroethoxy, —NH₂, —N(CH₃)₂, cyclopropylamino, —N(CH₃)-cyclobutyl, —N(CH₃)-methylthiazolyl, methylpiperidylamino, —NH—(C1-C3)-alkyl-N(CH₃)₂, morpholinyl, methylpiperazinyl, optionally methyl-substituted azetidinyl, thiomorpholinyl, optionally methyl-substituted hexahydropyrrolopyrrolyl, diazaspirononanyl, optionally substituted with methyl octahydropyrrolopyridinyl.

In another embodiment, the present invention includes a compound of Formula (Ia):

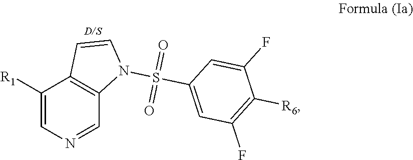

or a pharmaceutically acceptable salt or solvate or hydrate thereof, wherein:
R₁ as defined above,
R₆ is chosen independently and is:
   —OH, —SH, —(C1-C3)-alkoxy, 3-7 membered heterocyclyl containing 0-1 oxygen atoms and/or 0-1 sulfur atom, (C3-C7)-cycloalkyl, —S—(C1-C3)-alkyl, (C3-C7)-cycloalkyl-S—(C1-C3)-alkyl, wherein each of the above groups is optionally partially or completely substituted with substituents selected from —OH, 1-4 halogen atoms, (C1-C3)-alkoxy, (C3-C7)-cycloalkyl, 3-7 membered heterocycle containing one oxygen atom, —NH₂, (C1-C3)-alkylamino, di(C1-C3)-alkylamino or 3-7-membered heterocycle containing one nitrogen atom and optionally 1-2 heteroatoms selected from oxygen and sulfur, or
   —NRaRb, where each Ra and Rb:
   is independently hydrogen, (C3-C7)-cycloalkyl, —(C1-C3)-alkyl-N(CH₃)₂, (C1-C3)-alkyl, partially or fully halogenated (C1-C3)-alkyl, 3-7 membered heterocycle containing 0-1 oxygen, 0-1 sulfur and/or 1-4 nitrogen, optionally substituted with 1-2 substituents selected from (C1-C3)-alkyl or partially or fully halogenated (C1-C3)-alkyl, 5-7 membered heteroaryl containing 0-1 oxygen, 0-1 sulfur and/or 1-4 nitrogen, said heteroaryl being optionally substituted with 1-2 substituents selected from (C1-C3)-alkyl or partially or fully halogenated (C1-C3)-alkyl,
alternatively Ra and Rb together with the nitrogen atom to which they are attached form a 4-7 membered heterocyclyl containing 1-2 nitrogen atoms and 0-1 oxygen atoms and/or 0-1 sulfur atoms, optionally substituted with 1-2 substituents selected from (C1-C3)-alkyl, partially or fully halogenated (C1-C3)-alkyl, or
alternatively, Ra and Rb, together with the nitrogen atom to which they are attached, form a 6-14-membered bicyclic heterocyclyl containing 1-2 nitrogen atoms and 0-1 oxygen and/or 0-1 sulfur atoms, optionally substituted with 1-4 substituents selected from halogen, (C1-C3)-alkyl, partially or fully halogenated (C1-C3)-alkyl, (C2-C3)-alkenyl, (C2-C3) alkynyl or (C1-C3) alkoxy.

In another embodiment, the present invention includes a compound of Formula (IIa):

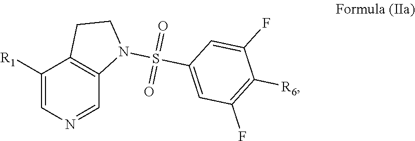

or a pharmaceutically acceptable salt, solvate or hydrate thereof, wherein:
R₁ and R₆ as defined above.

In another embodiment, the present invention includes a compound of Formula (Ib):

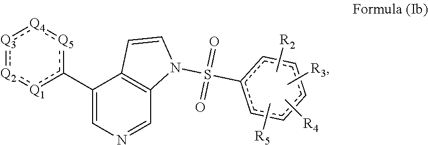

or a pharmaceutically acceptable salt, solvate or hydrate thereof, wherein:
Q₁, Q₂, Q₃, Q₄, Q₅ are each independently selected and are nitrogen or carbon, where no more than two of Q₁, Q₂, Q₃, Q₄, and Q₅ are nitrogen, and said carbon is optionally substituted with 1-5 substituents R1a,
R₂, R₃, R₄, R₅ and R1a as defined above,
dotted bonds mean the rings are aromatic.

In another embodiment, the present invention includes a compound of Formula (Ic):

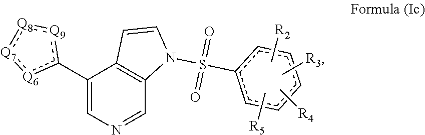

or a pharmaceutically acceptable salt, solvate or hydrate thereof, wherein:
Q₆, Q₇, Q₈, Q₉ are each independently selected and are oxygen, carbon, or nitrogen, with no more than two of Q₆, Q₇, Q₈ and Q₉ being nitrogen or oxygen, and said carbon is optionally substituted with 1-2 R1a groups,
R₂, R₃, R₄, R₅ and R1a as defined above,
dotted bonds mean the rings are aromatic.

In another embodiment, the present invention includes a compound of Formula (Id):

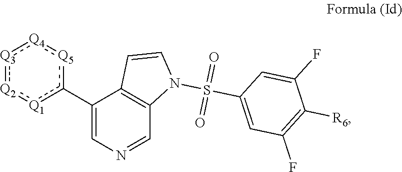

or a pharmaceutically acceptable salt, solvate or hydrate thereof, wherein:
Q₁, Q₂, Q₃, Q₄, Q₅ and R₆ as defined above.

In another embodiment, the present invention includes a compound of Formula (Ie):

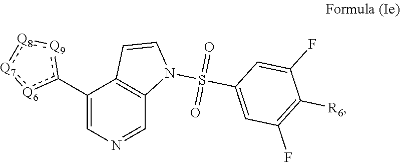

or a pharmaceutically acceptable salt, solvate or hydrate thereof, wherein:
Q₆, Q₇, Q₈, Q₉ and R₆ as defined above.

In another embodiment, the present invention includes a compound of Formula (If):

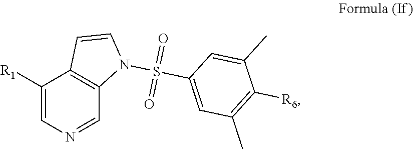

or a pharmaceutically acceptable salt, solvate or hydrate thereof, wherein:
R₁ and R₆ as defined above.

In another embodiment, the present invention includes a compound of Formula (IIb):

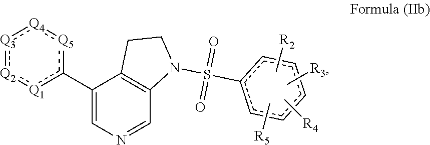

or a pharmaceutically acceptable salt, solvate or hydrate thereof, wherein:
Q₁, Q₂, Q₃, Q₄, Q₅, R₂, R₃, R₄ and R₅ as defined above;
dotted bonds mean the rings are aromatic.

In another embodiment, the present invention includes a compound of Formula (IId):

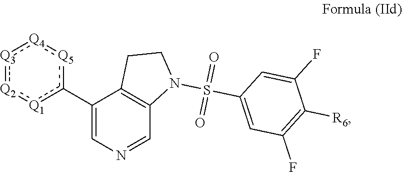

or a pharmaceutically acceptable salt, solvate or hydrate thereof, wherein:
Q₁, Q₂, Q₃, Q₄, Q₅ and R₆ as defined above.

In another embodiment, the present invention includes a compound of Formula (IIId):

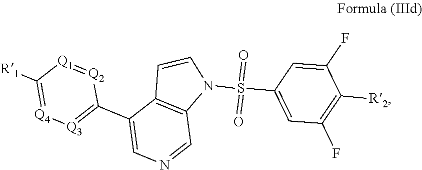

or a pharmaceutically acceptable salt, solvate or hydrate thereof, wherein:
Q₁, Q₂, Q₃, Q₄ are each independently represent nitrogen or carbon;
   if R′₁ independently represents hydrogen, deuterium, halogen, (C1-C3) alkyl, (C3-C5) cycloalkyl, —C(═O)—CH₃, partially or fully halogenated (C1-C3) alkyl, (C1-C3) alkylsulfonyl, cyano, N,N-dimethylamine, N-methylamine, then R′₂ independently represents 4-7-membered heterocyclyl containing 1-2 nitrogen atoms, 0-1 oxygen atom and/or 0-1 sulfur atom, necessary substituted 1-2 substituents selected from oxo group or —(C1-C3)-alkyl-(C3-C6)-cycloalkyl, and also optionally substituted with 1-3 substituents selected from deuterium, halogen, (C1-C5)-alkyl, partially or fully halogenated (C1-C5) alkyl, (C2-C3) alkenyl, (C2-C3) alkynyl and (C1-C3) alkoxy; or
   if R′₁ independently represents —C(═O)—NH—(C1-C5)-alkyl; then R₂ independently represents:
   NRcRd, where each Rc and Rd:
   is independently hydrogen, deuterium, (C3-C7)-cycloalkyl, —(C1-C3)-alkyl-N(CH₃)₂, (C1-C3)-alkyl, partially or fully halogenated (C1-C3)-alkyl, 3-7 membered heterocyclyl containing 0-1 oxygen atom, 0-1 sulfur atom and/or 0-4 nitrogen atoms, optionally substituted with 1-2 substituents selected from (C1-C3)-alkyl or partially or fully halogenated (C1-C3)-alkyl, 5-7-membered heteroaryl containing 0-1 oxygen atom, 0-1 sulfur atom and/or 0-4 nitrogen atoms, said heteroaryl being optionally substituted with 1-2 substituents selected from (C1-C3)-alkyl or partially or fully halogenated (C1-C3)-alkyl,
alternatively, Rc and Rd together with the nitrogen atom to which they are attached form a 4-7 membered heterocyclyl containing 1-2 nitrogen atoms and 0-1 oxygen atom and/or 0-1 sulfur atom, optionally substituted with 1-4 substituents selected from deuterium, halogen, (C1-C5)-alkyl, partially or fully halogenated (C1-C5)-alkyl, (C2-C3)-alkenyl, oxo group, —(C1-C3)-alkyl-(C3-C6)-cycloalkyl or (C1-C3) alkoxy, or
alternatively, Rc and Rd together with the nitrogen atom to which they are attached form a 6-14-membered bicyclic heterocyclyl containing 1-2 nitrogen atoms and 0-1 oxygen and/or 0-1 sulfur atoms, optionally substituted with 1-4 substituents selected from deuterium, halogen, (C1-C5)-alkyl, partially or fully halogenated (C1-C5)-alkyl, (C2-C3)-alkenyl, (C2-C3) alkynyl, oxo group or —(C1-C3)-alkyl-(C3-C6)-cycloalkyl or (C1-C3) alkoxy.

In a particular embodiment, the present invention includes compounds selected from the group (names given in accordance with IUPAC):
1_01 1-(4-methoxy-3,5-dimethylphenyl)sulfonyl-4-(5-methyl-2-pyridyl)pyrrolo[2,3-c]pyridine;
1_02 1-((4-methoxy-3,5-dimethylphenyl)sulfonyl)-4-(5-methylpyrazin-2-yl)-1H-pyrrolo[2,3-c]pyridine;
1_03 1-((4-methoxy-3,5-dimethylphenyl)sulfonyl)-4-(5-methylpyrimidin-2-yl)-1H-pyrrolo[2,3-c]pyridine;
1_04 1-(4-methoxy-3,5-dimethylphenyl)sulfonyl-4-(6-methylpyridazin-3-yl)pyrrolo[2,3-c]pyridine;
1_05 1-(4-methoxy-3,5-dimethylphenyl)sulfonyl-4-(5-methylsulfonyl-2-pyridyl)pyrrolo[2,3-c]pyridine;
1_06 1-((3,5-difluoro-4-isopropoxyphenyl)sulfonyl)-4-(pyridin-2-yl)-1H-pyrrolo[2,3-c]pyridine;
1_07 1-((4-(cyclopropylmethoxy)-3,5-difluorophenyl)sulfonyl)-4-(pyridin-2-yl)-1H-pyrrolo[2,3-c]pyridine;
1_08 1-((4-(2,2-difluoroethoxy)-3,5-difluorophenyl)sulfonyl)-4-(pyridin-2-yl)-1H-pyrrolo[2,3-c]pyridine;
1_09 2,6-difluoro-N,N-dimethyl-4-((4-(pyridin-2-yl)-1H-pyrrolo[2,3-c]pyridin-1-yl)sulfonyl)aniline;
1_10 4-(2,6-difluoro-4-((4-(pyridin-2-yl)-1H-pyrrolo[2,3-c]pyridin-1-yl)sulfonyl)phenyl)morpholine;
1_11 2,6-difluoro-4-((4-(pyridin-2-yl)-1H-pyrrolo[2,3-c]pyridin-1-yl)sulfonyl)aniline;
1_12 1-((3,5-difluoro-4-(4-methylpiperazin-1-yl)phenyl)sulfonyl)-4-(pyridin-2-yl)-1H-pyrrolo[2,3-c]pyridine;
1_13 N-cyclopropyl-2,6-difluoro-4-((4-(pyridin-2-yl)-1H-pyrrolo[2,3-c]pyridin-1-yl)sulfonyl)aniline;
1_14 N-cyclobutyl-2,6-difluoro-N-methyl-4-((4-(pyridin-2-yl)-1H-pyrrolo[2,3-c]pyridin-1-yl)sulfonyl)aniline;
1_15 2,6-difluoro-N,N-dimethyl-4-((4-(pyridin-2-yl)-2,3-dihydro-1H-pyrrolo[2,3-c]pyridin-1-yl)sulfonyl)aniline;
1_16 2,6-difluoro-4-((4-(pyridin-2-yl)-2,3-dehydro-H-pyrrolo[2,3-c]pyridin-1-yl)sulfonyl)phenol;
1_17 1-((3,5-difluoro-4-(4-methylpiperazin-1-yl)phenyl)sulfonyl)-4-(pyridin-2-yl)-2,3-dihydro-1H-pyrrolo[2,3-c]pyridine;
1_18 1-((3,5-difluoro-4-methoxyphenyl)sulfonyl)-4-(pyridin-2-yl)-1H-pyrrolo[2,3-c]pyridine;
1_19 1-((3,5-difluoro-4-(3-methylazetidin-1-yl)phenyl)sulfonyl)-4-(pyridin-2-yl)-1H-pyrrolo[2,3-c]pyridine;
1_20 N-(2,6-difluoro-4-((4-(pyridin-2-yl)-1H-pyrrolo[2,3-c]pyridin-1-yl)sulfonyl)phenyl)-N,4-dimethylthiazol-2-amine;
1_21 N-(2,6-difluoro-4-((4-(pyridin-2-yl)-1H-pyrrolo[2,3-c]pyridin-1-yl)sulfonyl)phenyl)-1-methylpiperidin-4-amine;
1_22 N1-(2,6-difluoro-4-((4-(pyridin-2-yl)-1H-pyrrolo[2,3-c]pyridin-1-yl)sulfonyl)phenyl)-N2,N2-dimethylethane-1,2-diamine;
1_23 4-(2,6-difluoro-4-((4-(pyridin-2-yl)-1H-pyrrolo[2,3-c]pyridin-1-yl)sulfonyl)phenyl)thiomorpholine;
1_24 1-((3,5-difluoro-4-(5-methylhexahydropyrrolo[3,4-c]pyrrol-2(1H)-yl)phenyl)sulfonyl)-4-(pyridin-2-yl)-1H-pyrrolo [2,3-s]pyridine;
1_25 1-((3,5-difluoro-4-(5-methyloctahydro-2H-pyrrolo[3,4-c]pyridin-2-yl)phenyl)sulfonyl)-4-(pyridin-2-yl)-1H-pyrrolo[2,3-c]pyridine;
1_26 2-(2,6-difluoro-4-((4-(pyridin-2-yl)-1H-pyrrolo[2,3-c]pyridin-1-yl)sulfonyl)phenyl)-2,7-diazaspiro[4.4]nonane;
1_27 2-(1-((3,5-difluoro-4-(4-methylpiperazin-1-yl)phenyl)sulfonyl)-1H-pyrrolo[2,3-c]pyridin-4-yl)oxazol-4-carbonitrile;
1_28 5-(1-((3,5-difluoro-4-(4-methylpiperazin-1-yl)phenyl)sulfonyl)-1H-pyrrolo[2,3-c]pyridin-4-yl)picolinonitrile;
1_29 1-((3,5-difluoro-4-(4-methylpiperazin-1-yl)phenyl)sulfonyl)-4-(5-fluoropyrimidin-2-yl)-1H-pyrrolo[2,3-c]pyridine;
1_30 5-(1-((3,5-difluoro-4-(4-methylpiperazin-1-yl)phenyl)sulfonyl)-1H-pyrrolo[2,3-c]pyridin-4-yl)-N-methylpyrazin-2-amine;
1_31 4-(5-cyclopropylpyridin-2-yl)-1-((3,5-difluoro-4-(4-methylpiperazin-1-yl)phenyl)sulfonyl)-1H-pyrrolo[2,3-c]pyridine;
1_32 2,6-difluoro-4-((4-(5-methylpyrazin-2-yl)-1H-pyrrolo[2,3-c]pyridin-1-yl)sulfonyl)aniline;
1_33 2,6-difluoro-N,N-dimethyl-4-((4-(5-methylpyridin-2-yl)-1H-pyrrolo[2,3-c]pyridin-1-yl)sulfonyl)aniline;
2_01 4-(2,6-difluoro-4-((4-(pyridin-2-yl)-1H-pyrrolo[2,3-c]pyridin-1-yl)sulfonyl)phenyl)-1-methylpiperazin-2-one;
2_02 1-((4-(4-(cyclopropylmethyl)piperazin-1-yl)-3,5-difluorophenyl)sulfonyl)-4-(pyridin-2-yl)-1H-pyrrolo[2,3-c]pyridine;
2_03 N-(tert-butyl)-5-(1-((3,5-difluoro-4-(5-methylhexahydropyrrolo[3,4-c]pyrrol-2(1H)-yl)phenyl)sulfonyl)-1H-pyrrolo[2,3-c]pyridin-4-yl)picolinamide;
3_01 1-((3,5-difluoro-4-(piperazin-1-yl)phenyl)sulfonyl)-4-(pyridin-2-yl)-1H-pyrrolo[2,3-c]pyridine;
3_02 6-(1-((3,5-difluoro-4-(4-methylpiperazin-1-yl)phenyl)sulfonyl)-1H-pyrrolo[2,3-c]pyridin-4-yl)-N-methylpyridin-3-amine;
3_03 5-(1-((3,5-difluoro-4-(4-methylpiperazin-1-yl)phenyl)sulfonyl)-1H-pyrrolo[2,3-c]pyridin-4-yl)-N-methylpyridin-2-amine;
3_04 6-(1-((3,5-difluoro-4-(4-methylpiperazin-1-yl)phenyl)sulfonyl)-1H-pyrrolo[2,3-c]pyridin-4-yl)-N,N-dimethylpyridin-3-amine;
3_05 5-(1-((3,5-difluoro-4-(4-methylpiperazin-1-yl)phenyl)sulfonyl)-1H-pyrrolo[2,3-c]pyridin-4-yl)pyrazine-2-carbonitrile;
3_06 6-(1-((3,5-difluoro-4-(4-methylpiperazin-1-yl)phenyl)sulfonyl)-1H-pyrrolo[2,3-c]pyridin-4-yl)nicotinonitrile;
3_07 6-(1-((3,5-difluoro-4-(4-methylpiperazin-1-yl)phenyl)sulfonyl)-1H-pyrrolo[2,3-c]pyridin-4-yl)-N-methylpyridazin-3-amine;
3_08 2-(1-((3,5-difluoro-4-(4-methylpiperazin-1-yl)phenyl)sulfonyl)-1H-pyrrolo[2,3-c]pyridin-4-yl)-N-methylpyrimidin-5-amine;
3_09 5-(1-((3,5-difluoro-4-(4-methylpiperazin-1-yl)phenyl)sulfonyl)-1H-pyrrolo[2,3-c]pyridin-4-yl)pyrimidine-2-carbonitrile;
3_10 6-(1-((3,5-difluoro-4-(4-methylpiperazin-1-yl)phenyl)sulfonyl)-1H-pyrrolo[2,3-c]pyridin-4-yl)pyridazine-3-carbonitrile;
3_11 1-((3,5-difluoro-4-(4-isopropylpiperazin-1-yl)phenyl)sulfonyl)-4-(pyridin-2-yl)-1H-pyrrolo[2,3-c]pyridine;
3_12 1-((4-(4-cyclopropylpiperazin-1-yl)-3,5-difluorophenyl)sulfonyl)-4-(pyridin-2-yl)-1H-pyrrolo[2,3-c]pyridine;
3_13 1-((3,5-difluoro-4-(4-methyl-1,4-diazepan-1-yl)phenyl)sulfonyl)-4-(pyridin-2-yl)-1H-pyrrolo[2,3-c]pyridine;
3_14 1-((4-(1,4-diazepan-1-yl)-3,5-difluorophenyl)sulfonyl)-4-(pyridin-2-yl)-1H-pyrrolo[2,3-c]pyridine;
3_15 1-(2,6-difluoro-4-((4-(pyridin-2-yl)-1H-pyrrolo[2,3-c]pyridin-1-yl)sulfonyl)phenyl)piperidin-4-amine;
3_16 1-((3,5-difluoro-4-(6-methyl-2,6-diazaspiro[3.3]heptan-2-yl)phenyl)sulfonyl)-4-(pyridin-2-yl)-1H-pyrrolo[2,3-c]pyridine;
3_17 1-((3,5-difluoro-4-(5-methyl-2,5-diazabicyclo[2.2.1]heptan-2-yl)phenyl)sulfonyl)-4-(pyridin-2-yl)-1H-pyrrolo[2,3-c]pyridine;
3_18 6-(2,6-difluoro-4-((4-(pyridin-2-yl)-1H-pyrrolo[2,3-c]pyridin-1-yl)sulfonyl)phenyl)-3-methyl-3,6-diazabicyclo[3.1.1]heptane;
3_19 1-((3,5-difluoro-4-(3-methyl-3,8-diazabicyclo[3.2.1]octan-8-yl)phenyl)sulfonyl)-4-(pyridin-2-yl)-1H-pyrrolo[2,3-c]pyridine;
3_20 1-((4-(1,4-diazabicyclo[3.2.1]octan-4-yl)-3,5-difluorophenyl)sulfonyl)-4-(pyridin-2-yl)-1H-pyrrolo[2,3-c]pyridine;
3_21 1-((3,5-difluoro-4-(2,6-diazaspiro[3.3]heptan-2-yl)phenyl)sulfonyl)-4-(pyridin-2-yl)-1H-pyrrolo[2,3-c]pyridine;
3_22 5-(1-((3,5-difluoro-4-(5-methylhexahydropyrrolo[3,4-c]pyrrol-2(1H)-yl)phenyl)sulfonyl)-1H-pyrrolo[2,3-c]pyridin-4-yl)-N-methylpyrazin-2-amine;
3_23 5-(1-((3,5-difluoro-4-(5-methylhexahydropyrrolo[3,4-c]pyrrol-2(1H)-yl)phenyl)sulfonyl)-1H-pyrrolo[2,3-c]pyridin-4-yl)pyrazine-2-carbonitrile;
3_24 5-(1-((3,5-difluoro-4-(5-methylhexahydropyrrolo[3,4-c]pyrrol-2(1H)-yl)phenyl)sulfonyl)-1H-pyrrolo[2,3-c]pyridin-4-yl)picolinonitrile;
3_25 1-((4-(2,5-diazabicyclo[2.2.1]heptan-2-yl)-3,5-difluorophenyl)sulfonyl)-4-(pyridin-2-yl)-1H-pyrrolo[2,3-c]pyridine (HCl salt);
3_26 1-((4-(3,8-diazabicyclo[3.2.1]octan-8-yl)-3,5-difluorophenyl)sulfonyl)-4-(pyridin-2-yl)-1H-pyrrolo[2,3-c]pyridine;
3_27 1-((3,5-difluoro-4-(piperazin-1-yl)phenyl)sulfonyl)-4-(pyridin-2-yl)-1H-pyrrolo[2,3-c]pyridine (HCl salt);
3_28 5-(1-((3,5-difluoro-4-(hexahydropyrrolo[3,4-c]pyrrol-2(1H)-yl)phenyl)sulfonyl)-1H-pyrrolo[2,3-c]pyridin-4-yl)picolinonitrile;
4_01 1-((3,5-difluoro-4-(4-methylpiperazin-1-yl)phenyl)sulfonyl)-4-(pyridin-2-yl)-1H-pyrrolo[2,3-c]pyridine (HCl salt).

The compounds of the invention may exist as various stereoisomers, diastereomers or enantiomers or mixtures thereof. All of these forms are part of the invention.

This invention also relates to the use of the compounds of the invention for the inhibition of CYP11B2.

The present invention also relates to the use of the compounds of the invention to lower the level of aldosterone in a subject.

In particular embodiments of the invention, the subject is a human.

The present invention also relates to the use of the compounds of the invention in the manufacture of CYP11 B2 inhibitor.

The present invention also relates to the use of the compounds of the invention in the manufacture of agent for the lowering the level of aldosterone in a subject.

The present invention also relates to the use of the compounds of the invention in the manufacture of a pharmaceutical composition for the treatment and/or prevention of a disease or condition that is mediated or maintained by CYP11 B2 activity, comprising a therapeutically or prophylactically effective amount of a compound of the invention and at least one pharmaceutically acceptable excipient.

The invention also includes a pharmaceutical composition for the treatment and/or prevention of a disease or condition that is mediated or maintained by CYP11 B2 activity, comprising a therapeutically or prophylactically effective amount of a compound of the invention and at least one pharmaceutically acceptable excipient.

In particular embodiments of the invention, the pharmaceutically acceptable excipient is a carrier, adjuvant and/or solvent.

In particular embodiments of the invention, the disease is selected from the group including primary hyperaldosteronism (Conn's syndrome), chronic heart failure, chronic heart failure with preserved ejection fraction, left ventricular dysfunction, left ventricular hypertrophy, arterial hypertension, resistant arterial hypertension, pulmonary arterial hypertension, coronary heart disease, liver cirrhosis, metabolic syndrome, chronic kidney disease, glomerulosclerosis, glomerulonephritis, nephritic syndrome, focal segmental glomerulosclerosis, diabetic nephropathy, heart and vascular remodeling, postinfarction cardiosclerosis, atherosclerosis, increased collagen formation, endothelial dysfunction, hypokalemia, or insulin resistance.

In particular embodiments of the invention, primary hyperaldosteronism is Conn's syndrome, including the following diseases or conditions: aldosterone-producing adenoma, idiopathic hyperaldosteronism, unilateral or bilateral adrenal hyperplasia, familial hyperaldosteronism of the first and second type, aldosterone-producing carcinoma, and aldosteronectopic.

The invention also relates to a method for lowering aldosterone levels in a subject, comprising administering to the subject a therapeutically effective amount of a compound of the invention.

The invention also includes a method for treating and/or preventing a disease or condition that is mediated or maintained by CYP11 B2 activity in a subject, comprising administering a therapeutically or prophylactically effective amount of a compound of the invention.

In particular embodiments of the invention, the disease or condition is selected from the group including primary hyperaldosteronism (Conn's syndrome), chronic heart failure, chronic heart failure with preserved ejection fraction, left ventricular dysfunction, left ventricular hypertrophy, arterial hypertension, resistant arterial hypertension, pulmonary arterial hypertension, coronary heart disease, liver cirrhosis, metabolic syndrome, chronic kidney disease, glomerulosclerosis, glomerulonephritis, nephritic syndrome, focal segmental glomerulosclerosis, diabetic nephropathy, cardiac and vascular remodeling, postinfarction cardiosclerosis, atherosclerosis, increased collagen formation, endothelial dysfunction, hypokalemia, or insulin resistance.

In particular embodiments of the invention, primary hyperaldosteronism is Conn's syndrome, including the following diseases or conditions: aldosterone-producing adenoma, idiopathic hyperaldosteronism, unilateral or bilateral adrenal hyperplasia, familial hyperaldosteronism of the first and second type, aldosterone-producing carcinoma, and aldosteronectopic.

In particular embodiments of the invention, the subject is a human.

The technical result of the present invention is to develop chemical compounds that are highly effective in inhibiting the activity of human cytochrome 11 B2 (CYP11 B2). These compounds are promising for the prevention and/or therapy of diseases or pathological conditions or disorders that are mediated or maintained by hypersynthesis of aldosterone.

### DETAILED DISCLOSURE OF THE INVENTION

### Definitions (Terms)

For a better understanding of the present invention, below are some of the terms used in the present description of the invention. The following definitions apply in this document unless otherwise stated. In addition, unless otherwise indicated, all occurrences of functional groups are selected independently, which may be indicated by the use of a slash to determine that two occurrences can be either the same or different (R₂, etc.). In the description of the present invention, the terms “comprises” and “comprising” are interpreted to mean “includes, among other things.” These terms are not intended to be construed as “consisting only of”.

The term “alkyl” as used herein, by itself or as part of another substituent, refers to straight or branched chain saturated hydrocarbon groups, including hydrocarbon groups having the indicated number of carbon atoms, refers to groups typically having from one to ten carbon atoms.

For example, the term (C1-C3)-alkyl means methyl, ethyl, propyl, etc.

The term “alkenyl” as used herein, refers to a linear or branched hydrocarbon-based aliphatic group comprising, unless otherwise mentioned, from 1 to 6 carbon atoms and at least an unsaturation. By way of examples, mention may be made of, but not limited to: vinyl group, and the like.

The term “alkynyl” as used herein, refers to a linear or branched hydrocarbon-based aliphatic group comprising, unless otherwise mentioned, from 1 to 6 carbon atoms and at least a triple bond. By way of examples, mention may be made of, but not limited to: acetylenyl group, and the like.

The term “partially or fully halogenated” includes branched or straight hydrocarbon chains in which one or more hydrogen atoms have been replaced by a halogen. Examples of haloalkyl groups include, but are not limited to, the following groups: trifluoromethyl, trichloromethyl, —C(CF₃)₂CH₃ and the like.

The term <<(C1-C3)-alkylsulfonyl>> means a —SO₂-alkyl where the alkyl group is as previously defined. By way of examples, mention may be made of, but not limited to SO₂CH₃, SO₂CH₂CH₃ and the like; The term “halogen” by itself or in part of another term refers to a fluorine, chlorine, bromine or iodine atom.

The term “aryl”, unless otherwise specified, means mono- or bicyclic aromatic rings containing 5-12 carbon atoms, in accordance with the Hückel aromaticity rule: a cyclic, planar molecule has 4n+2 π (Pi) electrons. Examples of aryl specifically include phenyl, naphthyl.

The term “alkoxy” refers to alkyl groups as defined above, which are attached to the molecule via a bridging oxygen atom. For example, the term “alkoxy” means —O-alkyl, where the alkyl group contains from 1 to 3 carbon atoms in the form of a linear (straight) or branched chain.

By way of illustration, alkoxy groups include, but are not limited to, the following groups: methoxy, ethoxy, n-propoxy, etc.

The term “alkylamino” refers to alkyl groups as defined above that are attached to the molecule via a bridging nitrogen atom. For example, the term “alkylamino” means —NH-alkyl or —N(-alkyl)₂, where the alkyl group contains from 1 to 3 carbon atoms in the form of a linear (straight) or branched chain. By way of illustration, alkylamino groups include, but are not limited to, the following groups: methylamino, N,N-dimethylamino, ethylamino, etc.

The term “bicyclic ring” or “bicyclic” as used herein means a bridged, fused or spiro-fused bicyclic ring system. Bicyclic heterocycles contain from 6 to 14 atoms, preferably 7 to 10 atoms. The bicyclic ring may contain one to four heteroatoms selected from the group consisting of nitrogen, oxygen and/or sulfur. Among bicyclic rings phenyl ring fused with 5-membered heterocycle may be cited.

Examples of a bicyclic structure include, but are not limited to, these examples:

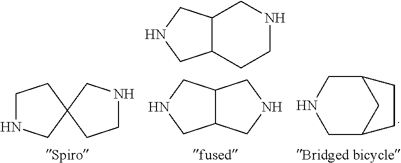

The term “heteroaryl” as used herein means a 5- or 7-membered aromatic ring according to Hückel's aromaticity rule: a cyclic, planar molecule has 4n+2 π (Pi) electrons containing from one to 5 heteroatoms selected from nitrogen atoms, oxygen and/or sulfur. Examples of heteroaryl include pyridinyl, pyrimidinyl, pyrazinyl, pyridazinyl, pyrazolyl, triazinyl, thiophenyl, furanyl, imidazolyl, oxazolyl, thiazolyl, isoxazolyl, isthiothiazolyl, triazolyl, tetrazolyl and the like.

The term “heterocycle” in the sense of the invention used here, means 3-7-membered non-aromatic ring containing from one to 4 heteroatoms selected from nitrogen, oxygen and/or sulfur atoms. The definition also includes partially saturated rings. Examples of the heterocycle include azepine, pyrrolidine, piperidine, oxirane, tetrahydrofuran, tetrahydropyran, oxepan, piperazine, morpholine, thiomorpholine, diazepine, oxazepane, thiazepine, homopiperazine, homomorpholine and the like. The term “heterocycle”, “heterocyclyl” or “heterocyclic” also refers to rings, saturated or partially unsaturated, which may be substituted.

This invention contains only such combinations of substituents and derivatives that form a stable or chemically possible compound. A stable or chemically possible compound is a compound whose stability is sufficient for its synthesis and analytical detection. Preferred compounds of this invention are sufficiently stable and do not decompose at temperatures up to 40° C. in the absence of reactive conditions, for at least one week.

Unless otherwise stated, the structures of the compounds given in the application materials also imply all stereoisomers, that is, the R- and S-isomers for each asymmetric center.

In addition, individual stereochemical isomers, as well as enantiomers and diastereomeric mixtures of the present compounds, are also the subject of this invention. Thus, the present invention embraces each diastereomer or enantiomer substantially free of other isomers (>90%; more preferably >95% molar purity), as well as a mixture of such isomers.

A particular optical isomer can be obtained by separating a racemic mixture according to a standard procedure, for example, by obtaining diastereomeric salts by treatment with an optically active acid or base, followed by separation of the mixture of diastereomers by crystallization, followed by isolating the optically active bases from these salts. Examples of suitable acids are tartaric, diacetyltartaric, dibenzoyltartaric, ditoluoyltartaric and camphorsulfonic acid. Another technique for separating optical isomers is to use a chiral chromatographic column.

In addition, another separation method involves the synthesis of covalent diastereomeric molecules by reacting compounds of the invention with an optically pure acid in activated form or an optically pure isocyanate. The resulting diastereomers can be separated by conventional means, such as chromatography, distillation, crystallization or sublimation, and then hydrolyzed to obtain an enantiomerically pure compound.

Optically active compounds of this invention can be obtained using optically active starting materials. Such isomers may be in free acid, free base, ester or salt form.

The term “solvate” refers to an association or complex of one or more solvent molecules and a compound of the invention. Examples of solvate-forming solvents include, but are not limited to, water, isopropanol, ethanol, methanol, DMSO, ethyl acetate, acetic acid, and ethanolamine.

The term “hydrate” refers to a complex where the solvent molecules are water.

The compounds of the invention may exist in a radiolabeled form, i. e. these compounds may contain one or more atoms whose atomic mass or mass number differs from the atomic mass or mass number of the most common natural isotopes. Radioisotopes of hydrogen, carbon, phosphorus, chlorine include ³H, ¹⁴C ³²P, ³⁵S, and ³⁶Cl, respectively. Compounds of this invention which contain such radioisotopes and/or other radioisotopes of other atoms are within the scope of the present invention. Tritium, i.e. ³H and carbon, i.e. ¹⁴C radioisotopes are particularly preferred due to their ease of preparation and detection.

Compounds of the present invention, labeled with radioactive isotopes, can be obtained using methods well known to experts in this field. Labeled compounds can be prepared using the procedures described here, by simply replacing unlabeled reagents with the appropriate labeled reagents.

The compounds of the present invention may exist in free form or, if required, in the form of a pharmaceutically acceptable salt or another derivative. The term “pharmaceutically acceptable salt” as used herein refers to those salts which, within the limits of medical judgment, are suitable for use in contact with human and animal tissues without undue toxicity, irritation, allergic reaction, etc., and meet a reasonable ratio of benefit. and risk. Pharmaceutically acceptable salts of amines, carboxylic acids, phosphonates and other types of compounds are well known in medicine. Salts can be obtained in situ during the isolation or purification of the compounds of the invention, and can also be obtained separately by reacting the free acid or free base of the compound of the invention with a suitable base or acid, respectively. Examples of pharmaceutically acceptable, non-toxic acid salts are amino salts formed with inorganic acids such as hydrochloric, hydrobromic, phosphoric, sulfuric and perchloric acids, or organic acids such as acetic, oxalic, maleic, tartaric, succinic or malonic acids, or obtained other methods used in this field, for example, using ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentane propionate, digluconate, dodecyl sulfate, ethanesulfonate, formate, fumarate, glucoheptonate, glycerophosphate, gluconate, hemisulfate, heptanate, hexanate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanate, valeriate and the like. Typical alkali and alkaline earth metal salts contain sodium, lithium, potassium, calcium, magnesium, and others. In addition, pharmaceutically acceptable salts may contain, if desired, non-toxic ammonium, quaternary ammonium and amine cations derived from counterions such as halides, hydroxides, carboxylates, sulfates, phosphates, nitrates, lower alkyl sulfonates and aryl sulfonates.

By “therapeutically effective amount” is meant that amount of a compound administered or delivered to a patient that is most likely to result in the desired response to treatment (prophylaxis) in the patient. The exact amount required may vary from subject to subject, depending on the age, body weight and general condition of the patient, the severity of the disease, the method of administration of the drug, combination treatment with other drugs, and the like.

The term “patient” (or “subject”) embraces all mammalian, preferably human, species that use the compounds of this invention, either by self-administration and/or administration to a patient by another person, for the treatment and/or prevention of a disease or medical condition.

The terms “treatment”, “therapy” cover the treatment of pathological conditions in mammals, preferably in humans, and include: a) blocking (suspension) of the course of the disease, b) alleviating the severity of the disease, i.e. induction of disease regression.

The term “prevention”, “preventive therapy” covers the elimination of risk factors, as well as the prophylactic treatment of subclinical stages of the disease in humans, aimed at reducing the likelihood of clinical stages of the disease. Patients for prophylactic therapy are selected on the basis of factors that, based on known data, entail an increased risk of clinical stages of the disease compared with the general population. Preventive therapy includes a) primary prevention and b) secondary prevention. Primary prevention is defined as prophylactic treatment in patients who have not yet reached the clinical stage of the disease. Secondary prevention is the prevention of the recurrence of the same or a close clinical condition of the disease.

The term “risk reduction” encompasses therapy that reduces the incidence of the clinical stage of the disease. Examples of disease risk reduction are primary and secondary disease prevention.

### IMPLEMENTATION OF THE INVENTION

### Examples

The specific compounds of the invention disclosed herein are provided for purposes of illustrating the present invention so that the invention may be more fully understood. Table 1 shows specific examples of compounds of the invention that can be prepared by the methods described in the General Synthesis Schemes, Examples, and known methods in the art. The methods listed are not exhaustive and are subject to reasonable modifications. These reactions must be carried out using suitable solvents and materials. When implementing these general procedures for the synthesis of specific substances, it is necessary to take into account the functional groups present in the substances and their influence on the course of the reaction. To obtain some substances, it is necessary to change the order of the stages or give preference to one of several alternative synthesis schemes. The compounds below are not to be considered as the only examples within the scope of the present invention, and in no way limit the invention.

In particular embodiments of the invention, the compounds of general Formula I are compounds N°1_01-4_01 listed in Table 1 below, as well as their pharmaceutically acceptable salts and/or stereoisomers.

In particular embodiments of the invention, the compounds of general Formula Ia are compounds N1_06-1_14, 1_18-4_01 listed in Table 1 below, as well as their pharmaceutically acceptable salts and/or stereoisomers.

In particular embodiments of the invention, the compounds of general Formula IIa are compounds N1_15-1_17 listed in Table 1 below, as well as their pharmaceutically acceptable salts and/or stereoisomers.

In particular embodiments of the invention, the compounds of general Formula IIb are compounds N°1_15-1_17 listed in Table 1 below, as well as their pharmaceutically acceptable salts and/or stereoisomers.

In particular embodiments of the invention, the compounds of general Formula IId are compounds N°1_15-1_17 listed in Table 1 below, as well as their pharmaceutically acceptable salts and/or stereoisomers.

In particular embodiments of the invention, the compounds of general Formula Ia are compounds N°1_06-1_14, 1_18-1_26 and 1_28-1_33 listed in Table 1 below, as well as their pharmaceutically acceptable salts and/or stereoisomers.

In particular embodiments of the invention, the compounds of general Formula Ic are compounds N°1_27 listed in Table 1 below, as well as their pharmaceutically acceptable salts and/or stereoisomers.

In particular embodiments of the invention, the compounds of general Formula Ie are compounds N°1_06-1_14, 1_18-1_26 and 1_28-1_33 listed in Table 1 below, as well as their pharmaceutically acceptable salts and/or stereoisomers.

In particular embodiments of the invention, the compounds of general Formula Ic are compounds N°1_27 listed in Table 1 below, as well as their pharmaceutically acceptable salts and/or stereoisomers.

In particular embodiments of the invention, the compounds of general Formula 1f are compounds N°1_1-1_5 listed in Table 1 below, as well as their pharmaceutically acceptable salts and/or stereoisomers.

TABLE 1

| No | Structure | Name |
|---|---|---|
| 1_01 | 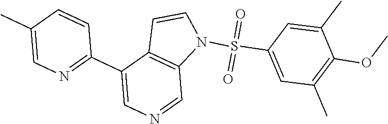 | 1-(4-methoxy-3,5-dimethylphenyl)sulfonyl- 4-(5-methyl-2-pyridyl)pyrrolo[2,3-c ]pyridine |
| 1_02 | 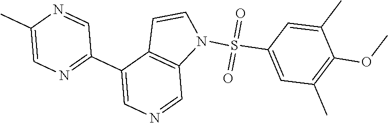 | 1-((4-Methoxy-3,5-dimethylphe- nyl)sulfonyl)-4-(5-methylpyrazin-2-yl)-1H- pyrrolo[2,3-c]pyridine |
| 1_03 | 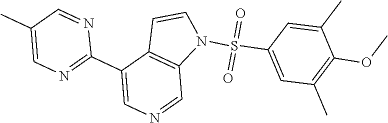 | 1-((4-methoxy-3,5-dimethylphenyl)sulfo- nyl)-4-(5-methylpyrimidin-2-yl)-1H- pyrrolo[2,3-c]pyridine |
| 1_04 | 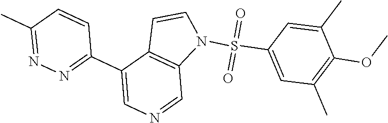 | 1-(4-methoxy-3,5-dimethylphenyl)sulfonyl- 4-(6-methylpyridazin-3-yl)pyrrolo[2,3- c]pyridine |
| 1_05 | 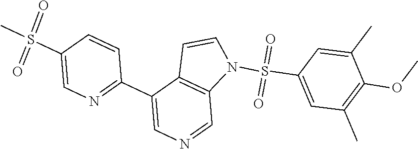 | 1-(4-methoxy-3,5-dimethylphenyl)sulfonyl- 4-(5-methylsulfonyl-2-pyridyl)pyrrolo[2,3- c]pyridine |
| 1_06 | 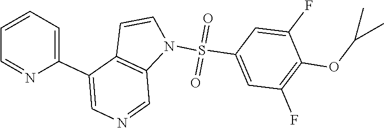 [SMILES: CC(C)Oc1c(F)cc(S(=O)(=O)n2ccc3c(-c4ccccn4)cncc32)cc1F] | 1-((3,5-difluoro-4-isopropoxyphenyl)sulfo- nyl)-4-(pyridin-2-yl)-1H-pyrrolo[2,3- c]pyridine |
| 1_07 | 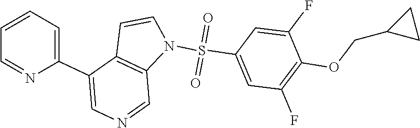 [SMILES: O=S(=O)(c1cc(F)c(OCC2CC2)c(F)c1)n1ccc2c(-c3ccccn3)cncc21] | 1-((4-(cyclopropylmethoxy)-3,5- difluorophenyl)sulfonyl)-4-(pyridin-2-yl)- 1H-pyrrolo[2,3-c]pyridine |
| 1_08 | 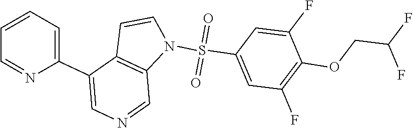 [SMILES: O=S(=O)(c1cc(F)c(OCC(F)F)c(F)c1)n1ccc2c(-c3ccccn3)cncc21] | 1-((4-(2,2-difluoroethoxy)-3,5- difluorophenyl)sulfonyl)-4-(pyridin-2-yl)- 1H-pyrrolo[2,3-c]pyridine |
| 1_09 | 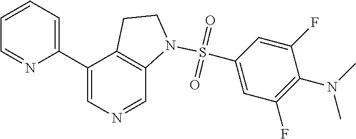 [SMILES: CN(C)c1c(F)cc(S(=O)(=O)N2CCc3c(-c4ccccn4)cncc32)cc1F] | 2,6-difluoro-N,N-dimethyl-4-((4-(pyridin-2- yl)-1H-pyrrolo[2,3-c]pyridin-1- yl)sulfonyl)aniline |
| 1_10 | 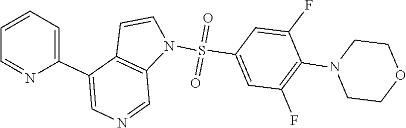 [SMILES: O=S(=O)(c1cc(F)c(N2CCOCC2)c(F)c1)n1ccc2c(-c3ccccn3)cncc21] | 4-(2,6-difluoro-4-((4-(pyridin-2-yl)-1H- pyrrolo[2,3-c]pyridin-1- yl)sulfonyl)phenyl)morpholine |
| 1_11 | 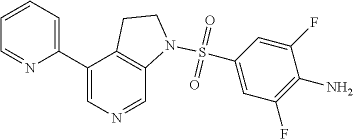 [SMILES: Nc1c(F)cc(S(=O)(=O)N2CCc3c(-c4ccccn4)cncc32)cc1F] | 2,6-difluoro-4-((4-(pyridin-2-yl)-1H-pyrrolo c[2,3-]pyridin-1-yl)sulfonyl)aniline |
| 1_12 | 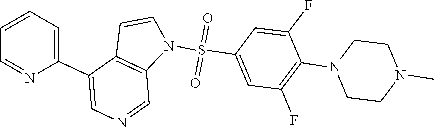 [SMILES: CN1CCN(c2c(F)cc(S(=O)(=O)n3ccc4c(-c5ccccn5)cncc43)cc2F)CC1] | 1-((3,5-difluoro-4-(4-methylpiperazin-1- yl)phenyl)sulfonyl)-4-(pyridin-2-yl)-1H- pyrrolo[2,3-c]pyridine |
| 1_13 | 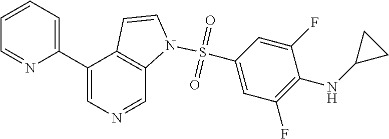 [SMILES: O=S(=O)(c1cc(F)c(NC2CC2)c(F)c1)n1ccc2c(-c3ccccn3)cncc21] | N-cyclopropyl-2,6-difluoro-4-((4-(pyridin- 2-yl)-1H-pyrrolo[2,3-c]pyridin-1-yl)sulfo- nyl)aniline |
| 1_14 | 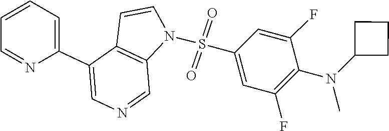 [SMILES: CN(c1c(F)cc(S(=O)(=O)n2ccc3c(-c4ccccn4)cncc32)cc1F)C1CCC1] | N-cyclobutyl-2,6-difluoro-N-methyl-4-((4- (pyridin-2-yl)-1H-pyrrolo[2,3-c]pyridin-1- yl)sulfonyl)aniline |
| 1_15 | 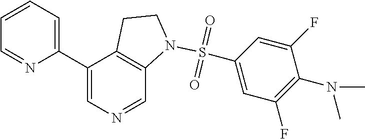 [SMILES: CN(C)c1c(F)cc(S(=O)(=O)N2CCc3c(-c4ccccn4)cncc32)cc1F] | 2,6-difluoro-N,N-dimethyl-4-((4-(pyridin-2- yl)-2,3-dihydro-1H-pyrrolo[2,3-c]pyridin-1- yl)sulfonyl)aniline |
| 1_16 | 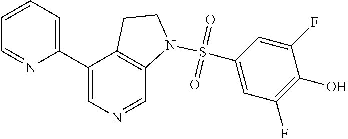 [SMILES: O=S(=O)(c1cc(F)c(O)c(F)c1)N1CCc2c(-c3ccccn3)cncc21] | 2,6-difluoro-4-((4-(pyridin-2-yl)-2,3- dehydro-H-pyrrolo[2,3-c]pyridin-1- yl)sulfonyl)phenol |
| 1_17 | 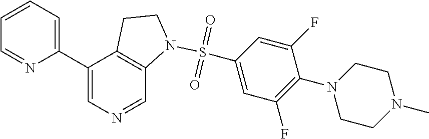 [SMILES: CN1CCN(c2c(F)cc(S(=O)(=O)N3CCc4c(-c5ccccn5)cncc43)cc2F)CC1] | 1-((3,5-difluoro-4-(4-methylpiperazin-1- yl)phenyl)sulfonyl)-4-(pyridin-2-yl)-2,3- dihydro-1H-pyrrolo[2,3-c]pyridine |
| 1_18 | 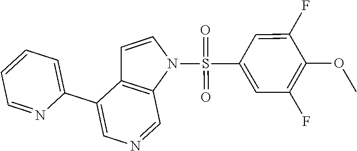 [SMILES: COc1c(F)cc(S(=O)(=O)n2ccc3c(-c4ccccn4)cncc32)cc1F] | 1-((3,5-difluoro-4-methoxyphenyl)sulfonyl)- 4-(pyridin-2-yl)-1H-pyrrolo[2,3-c]pyridine |
| 1_19 | 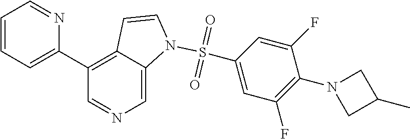 [SMILES: CC1CN(c2c(F)cc(S(=O)(=O)n3ccc4c(-c5ccccn5)cncc43)cc2F)C1] | 1-((3,5-difluoro-4-(3-methylazetidin-1- yl)phenyl)sulfonyl)-4-(pyridin-2-yl)-1H- pyrrolo[2,3-c]pyridine |
| 1_20 | 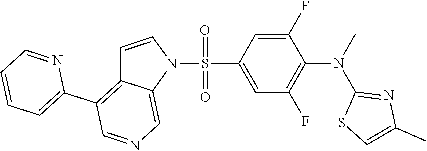 [SMILES: Cc1csc(N(C)c2c(F)cc(S(=O)(=O)n3ccc4c(-c5ccccn5)cncc43)cc2F)n1] | N-(2,6-difluoro-4-((4-(pyridin-2-yl)-1H- pyrrolo[2,3-c]pyridin-1-yl)sulfonyl)phenyl)- N,4-dimethylthiazol- 2-amine |
| 1_21 | 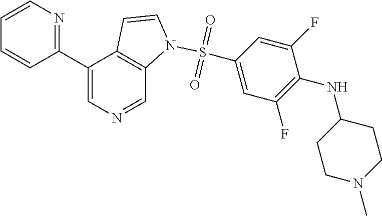 [SMILES: CN1CCC(Nc2c(F)cc(S(=O)(=O)n3ccc4c(-c5ccccn5)cncc43)cc2F)CC1] | N-(2,6-difluoro-4-((4-(pyridin-2-yl)-1H- pyrrolo[2,3-c]pyridin-1-yl)sulfonyl)phenyl)- 1-methylpiperidin-4-amine |
| 1_22 | 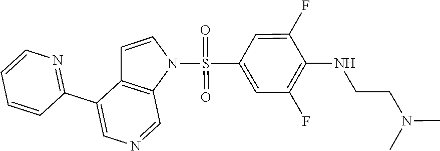 [SMILES: CN(C)CCNc1c(F)cc(S(=O)(=O)n2ccc3c(-c4ccccn4)cncc32)cc1F] | N1-(2,6-difluoro-4-((4-(pyridin-2-yl)-1H- pyrrolo[2,3-c]pyridin-1-yl)sulfonyl)phenyl)- N2,N2-dimethylethane-1,2-diamine |
| 1_23 | 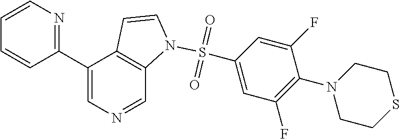 [SMILES: O=S(=O)(c1cc(F)c(N2CCSCC2)c(F)c1)n1ccc2c(-c3ccccn3)cncc21] | 4-(2,6-difluoro-4-((4-(pyridin-2-yl)-1H- pyrrolo[2,3-c]pyridin-1- yl)sulfonyl)phenyl)thiomorpholine |
| 1_24 | 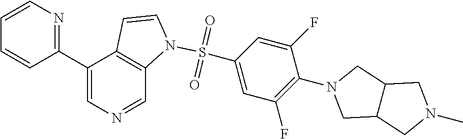 [SMILES: CN1CC2CN(c3c(F)cc(S(=O)(=O)n4ccc5c(-c6ccccn6)cncc54)cc3F)CC2C1] | 1-((3,5-difluoro-4-(5- methylhexahydropyrrolo[3,4-c]pyrrol-2(1H)- yl)phenyl)sulfonyl)-4-(pyridin-2-yl)-1H- pyrrolo[2,3-s]pyridine |
| 1_25 | 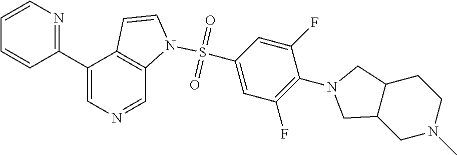 [SMILES: CN1CCC2CN(c3c(F)cc(S(=O)(=O)n4ccc5c(-c6ccccn6)cncc54)cc3F)CC2C1] | 1-((3,5-difluoro-4-(5-methyloctahydro-2H- pyrrolo[3,4-c]pyridin-2-yl)phenyl)sulfonyl)- 4-(pyridin-2-yl)-1H-pyrrolo[2,3-c]pyridine |
| 1_26 | 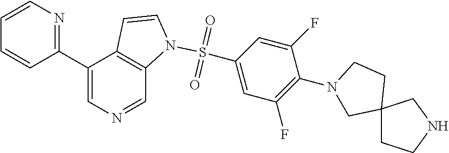 [SMILES: O=S(=O)(c1cc(F)c(N2CCC3(CCNC3)C2)c(F)c1)n1ccc2c(-c3ccccn3)cncc21] | 2-(2,6-difluoro-4-((4-(pyridin-2-yl)-1H- pyrrolo[2,3-c]pyridin-1-yl)sulfonyl)phenyl)- 2,7-diazaspiro[ 4.4]nonane |
| 1_27 | 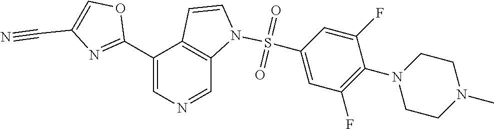 [SMILES: CN1CCN(c2c(F)cc(S(=O)(=O)n3ccc4c(-c5nc(C#N)co5)cncc43)cc2F)CC1] | 2-(1-((3,5-difluoro-4-(4-methylpiperazin-1- yl)phenyl)sulfonyl)-1H-pyrrolo[2,3-c]pyri- din-4-yl)oxazol-4-carbonitrile |
| 1_28 | 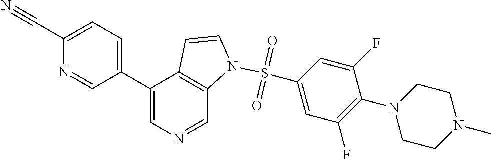 [SMILES: CN1CCN(c2c(F)cc(S(=O)(=O)n3ccc4c(-c5ccc(C#N)nc5)cncc43)cc2F)CC1] | 5-(1-((3,5-difluoro-4-(4-methylpiperazin-1- yl)phenyl)sulfonyl)-1H-pyrrolo[2,3-c]pyri- din-4-yl)picolinonitrile |
| 1_29 | 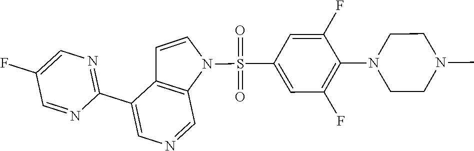 [SMILES: CN1CCN(c2c(F)cc(S(=O)(=O)n3ccc4c(-c5ncc(F)cn5)cncc43)cc2F)CC1] | 1-((3,5-difluoro-4-(4-methylpiperazin-1- yl)phenyl)sulfonyl)-4-(5-fluoropyrimidin-2- yl)-1H-pyrrolo[2,3-c]pyridine |
| 1_30 | 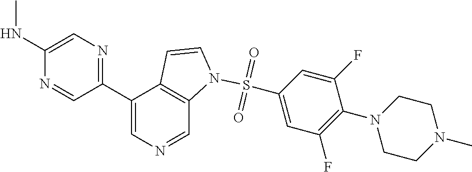 [SMILES: CNc1cnc(-c2cncc3c2ccn3S(=O)(=O)c2cc(F)c(N3CCN(C)CC3)c(F)c2)cn1] | 5-(1-((3,5-difluoro-4-(4-methylpiperazin-1- yl)phenyl)sulfonyl)-1H-pyrrolo[2,3- c]pyridin-4-yl)-N-methylpyrazin- 2-amine |
| 1_31 | 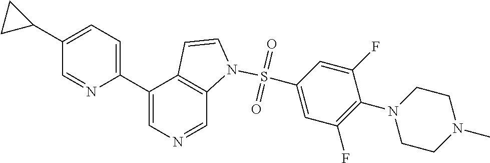 [SMILES: CN1CCN(c2c(F)cc(S(=O)(=O)n3ccc4c(-c5ccc(C6CC6)cn5)cncc43)cc2F)CC1] | 4-(5-cyclopropylpyridin-2-yl)-1-((3,5- difluoro-4-(4-methylpiperazin-1-yl)phe- nyl)sulfonyl)-1H-pyrrolo[2,3-c]pyridine |
| 1_32 | 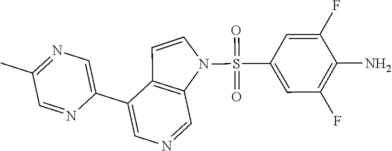 [SMILES: Cc1cnc(-c2cncc3c2ccn3S(=O)(=O)c2cc(F)c(N)c(F)c2)cn1] | 2,6-difluoro-4-((4-(5-methylpyrazin-2-yl)- 1H-pyrrolo[2,3-c]pyridin-1-yl)sulfo- nyl)aniline |
| 1_33 | 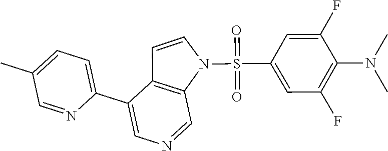 | 2,6-difluoro-N,N-dimethyl-4-((4-(5- methylpyridin-2-yl)-1H-pyrrolo[2,3-c]pyri- din-1-yl)sulfonyl)aniline |
| 2_01 | 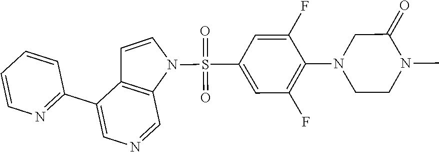 [SMILES: CN1CCN(c2c(F)cc(S(=O)(=O)n3ccc4c(-c5ccccn5)cncc43)cc2F)CC1=O] | 4-(2,6-difluoro-4-((4-(pyridin-2-yl)-1H- pyrrolo[2,3-c]pyridin-1-yl)sulfo- nyl)phenyl)-1-methylpiperazin-2-one |
| 2_02 | 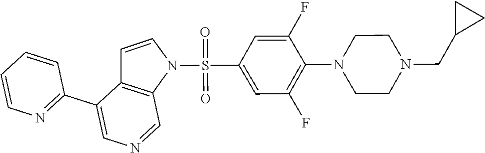 [SMILES: O=S(=O)(c1cc(F)c(N2CCN(CC3CC3)CC2)c(F)c1)n1ccc2c(-c3ccccn3)cncc21] | 1-((4-(4-(cyclopropylmethyl)piperazin-1- yl)-3,5-difluorophenyl)sulfonyl)-4- (pyridin-2-yl)-1H-pyrrolo[2,3-c]pyridine |
| 2_03 | 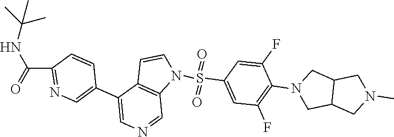 [SMILES: CN1CC2CN(c3c(F)cc(S(=O)(=O)n4ccc5c(-c6ccc(C(=O)NC(C)(C)C)nc6)cncc54)cc3F)CC2C1] | N-(tert-butyl)-5-(1-((3,5-difluoro-4-(5- methylhexahydropyrrolo[3,4-c]pyrrol-2 (1H)-yl)phenyl)sulfonyl)-1 H-pyrrolo[2,3- c]pyridin-4-yl)picolinamide |
| 3_01 | 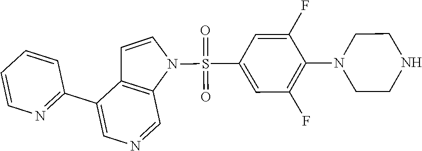 [SMILES: O=S(=O)(c1cc(F)c(N2CCNCC2)c(F)c1)n1ccc2c(-c3ccccn3)cncc21] | 1-((3,5-difluoro-4-(piperazin-1- yl)phenyl)sulfonyl)-4-(pyridin-2-yl)-1H- pyrrolo[2,3-c]pyridine |
| 3_02 | 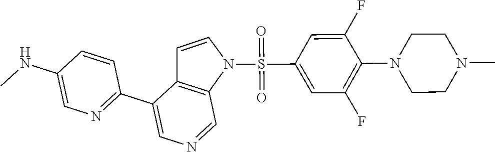 | 6-(1-((3,5-difluoro-4-(4-methylpiperazin-1- yl)phenyl)sulfonyl)-1H-pyrrolo[2,3-c]pyri- din-4-yl)-N-methylpyridin-3-amine |
| 3_03 | 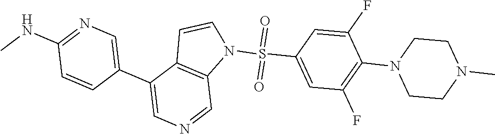 | 5-(1-((3,5-difluoro-4-(4-methylpiperazin-1- yl)phenyl)sulfonyl)-1H-pyrrolo[2,3-c]pyri- din-4-yl)-N-methylpyridin-2-amine |
| 3_04 | 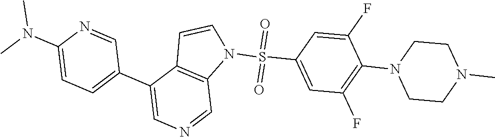 | 6-(1-((3,5-difluoro-4-(4-methylpiperazin-1- yl)phenyl)sulfonyl)-1H-pyrrolo[2,3-c]pyri- din-4-yl)-N,N-dimethylpyridin-3-amine |
| 3_05 | 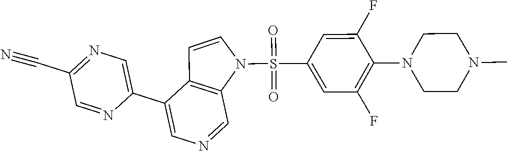 [SMILES: CN1CCN(c2c(F)cc(S(=O)(=O)n3ccc4c(-c5cnc(C#N)cn5)cncc43)cc2F)CC1] | 5-(1-((3,5-difluoro-4-(4-methylpiperazin-1- yl)phenyl)sulfonyl)-1H-pyrrolo[2,3-c]pyri- din-4-yl)pyrazine-2-carbonitrile |
| 3_06 | 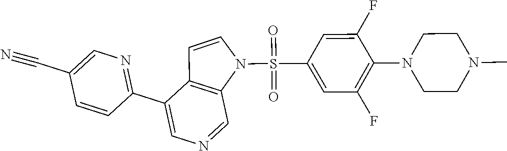 [SMILES: CN1CCN(c2c(F)cc(S(=O)(=O)n3ccc4c(-c5ccc(C#N)cn5)cncc43)cc2F)CC1] | 6-(1-((3,5-difluoro-4-(4-methylpiperazin-1- yl)phenyl)sulfonyl)-1H-pyrrolo[2,3-c]pyri- din-4-yl)nicotinonitrile |
| 3_07 | 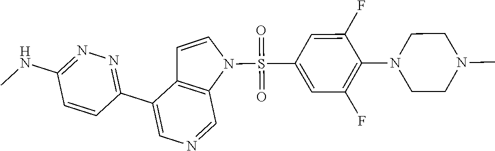 | 6-(1-((3,5-difluoro-4-(4-methylpiperazin-1- yl)phenyl)sulfonyl)-1H-pyrrolo[2,3-c]pyri- din-4-yl)-N-methylpyridazin-3-amine |
| 3_08 | 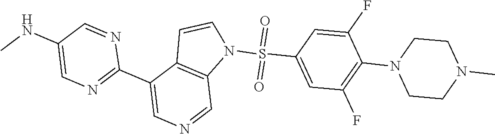 | 2-(1-((3,5-difluoro-4-(4-methylpiperazin-1- yl)phenyl)sulfonyl)-1H-pyrrolo[2,3-c]pyri- din-4-yl)-N-methylpyrimidin-5-amine |
| 3_09 | 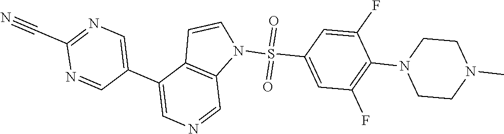 [SMILES: CN1CCN(c2c(F)cc(S(=O)(=O)n3ccc4c(-c5cnc(C#N)nc5)cncc43)cc2F)CC1] | 5-(1-((3,5-difluoro-4-(4-methylpiperazin-1- yl)phenyl)sulfonyl)-1H-pyrrolo[2,3-c]pyri- din-4-yl)pyrimidine-2-carbonitrile |
| 3_10 | 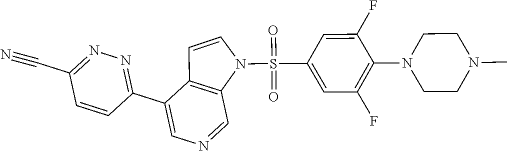 [SMILES: CN1CCN(c2c(F)cc(S(=O)(=O)n3ccc4c(-c5ccc(C#N)nn5)cncc43)cc2F)CC1] | 6-(1-((3,5-difluoro-4-(4-methylpiperazin-1- yl)phenyl)sulfonyl)-1H-pyrrolo[2,3-c]pyri- din-4-yl)pyridazine-3-carbonitrile |
| 3_11 | 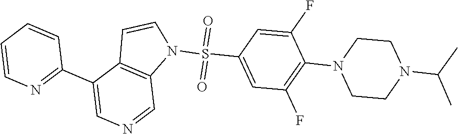 [SMILES: CC(C)N1CCN(c2c(F)cc(S(=O)(=O)n3ccc4c(-c5ccccn5)cncc43)cc2F)CC1] | 1-((3,5-difluoro-4-(4-isopropylpiperazin-1- yl)phenyl)sulfonyl)-4-(pyridin-2-yl)-1H- pyrrolo[2,3-c]pyridine |
| 3_12 | 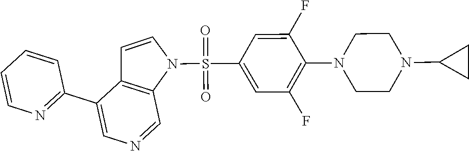 [SMILES: O=S(=O)(c1cc(F)c(N2CCN(C3CC3)CC2)c(F)c1)n1ccc2c(-c3ccccn3)cncc21] | 1-((4-(4-cyclopropylpiperazin-1-yl)-3,5- difluorophenyl)sulfonyl)-4-(pyridin-2-yl)- 1H-pyrrolo[2,3-c]pyridine |
| 3_13 | 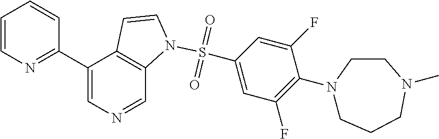 [SMILES: CN1CCCN(c2c(F)cc(S(=O)(=O)n3ccc4c(-c5ccccn5)cncc43)cc2F)CC1] | 1-((3,5-difluoro-4-(4-methyl-1,4-diazepan- y1-l)phenyl)sulfonyl)-4-(pyridin-2-yl)-1H- pyrrolo[2,3-c]pyridine |
| 3_14 | 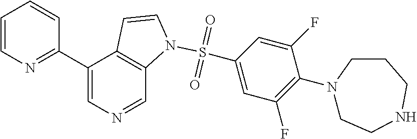 [SMILES: O=S(=O)(c1cc(F)c(N2CCCNCC2)c(F)c1)n1ccc2c(-c3ccccn3)cncc21] | 1-((4-(1,4-diazepan-1-yl)-3,5- difluorophenyl)sulfonyl)-4-(pyridin-2-yl)- 1H-pyrrolo[2,3-c]pyridine |
| 3_15 | 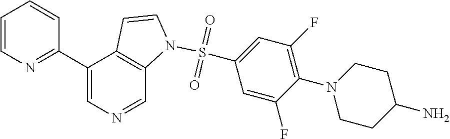 [SMILES: NC1CCN(c2c(F)cc(S(=O)(=O)n3ccc4c(-c5ccccn5)cncc43)cc2F)CC1] | 1-(2,6-difluoro-4-((4-(pyridin-2-yl)-1H- pyrrolo[2,3-c]pyridin-1- yl)sulfonyl)phenyl)piperidin-4-amine |
| 3_16 | 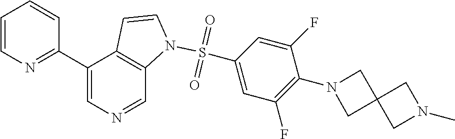 [SMILES: CN1CC2(C1)CN(c1c(F)cc(S(=O)(=O)n3ccc4c(-c5ccccn5)cncc43)cc1F)C2] | 1-((3,5-difluoro-4-(6-methyl-2,6-di- azaspiro[3.3]heptan-2-yl)phenyl)sulfonyl)- 4-(pyridin-2-yl)-1H-pyrrolo[2,3-c]pyridine |
| 3_17 | 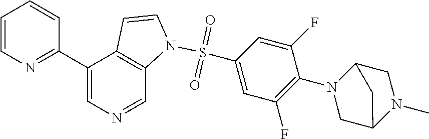 [SMILES: CN1CC2CC1CN2c1c(F)cc(S(=O)(=O)n2ccc3c(-c4ccccn4)cncc32)cc1F] | 1-((3,5-difluoro-4-(5-methyl-2,5- diazabicyclo[2.2.1]heptan-2- yl)phenyl)sulfonyl)-4-(pyridin-2-yl)-1H- pyrrolo[2,3-c]pyridine |
| 3_18 | 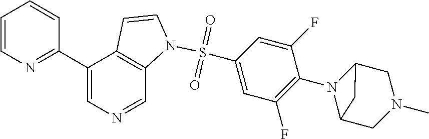 [SMILES: CN1CC2CC(C1)N2c1c(F)cc(S(=O)(=O)n2ccc3c(-c4ccccn4)cncc32)cc1F] | 6-(2,6-difluoro-4-((4-(pyridin-2-yl)-1H- pyrrolo[2,3-c]pyridin-1-yl)sulfo- nyl)phenyl)-3-methyl-3,6-diazabicy- clo[3.1.1]heptane |
| 3_19 | 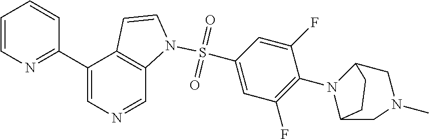 [SMILES: CN1CC2CCC(C1)N2c1c(F)cc(S(=O)(=O)n2ccc3c(-c4ccccn4)cncc32)cc1F] | 1-((3,5-difluoro-4-(3-methyl-3,8-diaza- bicyclo[3.2.1]octan-8-yl)phenyl)sulfonyl)- 4-(pyridin-2-yl)-1H-pyrrolo[2,3-c]pyridine |
| 3_20 | 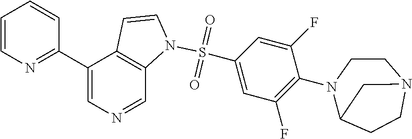 [SMILES: O=S(=O)(c1cc(F)c(N2CCN3CCC2C3)c(F)c1)n1ccc2c(-c3ccccn3)cncc21] | 1-((4-(1,4-diazabicyclo[3.2.1]octan-4-yl)- 3,5-difluorophenyl)sulfonyl)-4-(pyridin-2- yl)-1H-pyrrolo[2,3-c]pyridine |
| 3_21 | 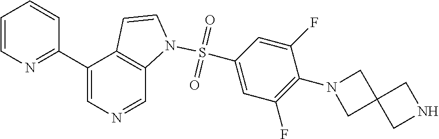 [SMILES: O=S(=O)(c1cc(F)c(N2CC3(CNC3)C2)c(F)c1)n1ccc2c(-c3ccccn3)cncc21] | 1-((3,5-difluoro-4-(2,6-diazaspiro[3.3]hep- tan-2-yl)phenyl)sulfonyl)-4-(pyridin-2-yl)- 1H-pyrrolo[2,3-c]pyridine |
| 3_22 | 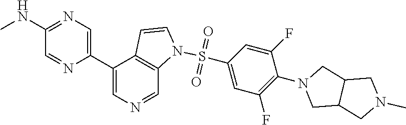 | 5-(1-((3,5-difluoro-4-(5-methyl- hexahydropyrrolo[3,4-c]pyrrol-2(1H)- yl)phenyl)sulfonyl)-1H-pyrrolo[2,3-c]pyri- din-4-yl)-N-methylpyrazin-2-amine |
| 3_23 | 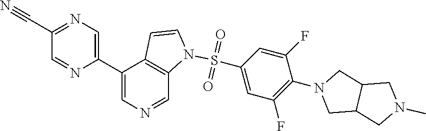 [SMILES: CN1CC2CN(c3c(F)cc(S(=O)(=O)n4ccc5c(-c6cnc(C#N)cn6)cncc54)cc3F)CC2C1] | 5-(1-((3,5-difluoro-4-(5-methyl- hexahydropyrrolo[3,4-c]pyrrol-2(1H)- yl)phenyl)sulfonyl)-1H-pyrrolo[2,3-c]pyri- din-4-yl)pyrazine-2-carbonitrile |
| 3_24 | 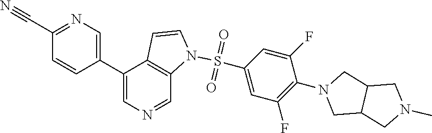 [SMILES: CN1CC2CN(c3c(F)cc(S(=O)(=O)n4ccc5c(-c6ccc(C#N)nc6)cncc54)cc3F)CC2C1] | 5-(1-((3,5-difluoro-4-(5-methyl- hexahydropyrrolo[3,4-c]pyrrol-2(1H)- yl)phenyl)sulfonyl)-1H-pyrrolo[2,3-c]pyri- din-4-yl)picolinonitrile |
| 3_25 | 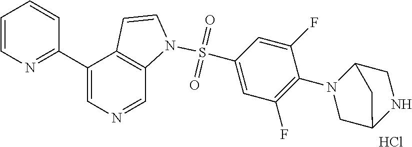 [SMILES: O=S(=O)(c1cc(F)c(N2CC3CC2CN3)c(F)c1)n1ccc2c(-c3ccccn3)cncc21 ; Cl] | 1-((4-(2,5-diazabicyclo[2.2.1]heptan-2-yl)- 3,5-difluorophenyl)sulfonyl)-4-(pyridin-2- yl)-1H-pyrrolo[2,3-c]pyridine (HCl salt) |
| 3_26 | 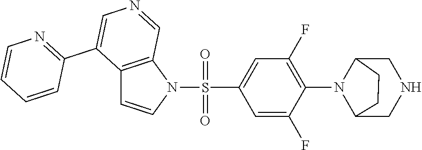 [SMILES: O=S(=O)(c1cc(F)c(N2C3CCC2CNC3)c(F)c1)n1ccc2c(-c3ccccn3)cncc21] | 1-((4-(3,8-diazabicyclo[3.2.1]octan-8-yl)- 3,5-difluorophenyl)sulfonyl)-4-(pyridin- 2-yl)-1H-pyrrolo[2,3-c]pyridine |
| 3_27 | 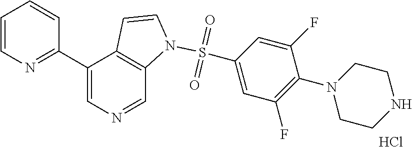 [SMILES: O=S(=O)(c1cc(F)c(N2CCNCC2)c(F)c1)n1ccc2c(-c3ccccn3)cncc21 ; Cl] | 1-((3,5-difluoro-4-(piperazin-1- yl)phenyl)sulfonyl)-4-(pyridin-2-yl)-1H- pyrrolo[2,3-c]pyridine (HCl salt) |
| 3_28 | 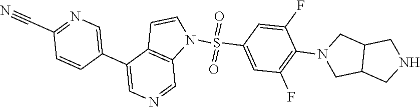 [SMILES: N#Cc1ccc(-c2cncc3c2ccn3S(=O)(=O)c2cc(F)c(N3CC4CNCC4C3)c(F)c2)cn1] | 5-(1-((3,5-difluoro-4-(hexahydropyrrolo [3,4-c]pyrrol-2(1H)-yl)phenyl)sulfonyl)- 1H-pyrrolo[2,3-c]pyridin-4- yl)picolinonitrile |
| 4_01 | 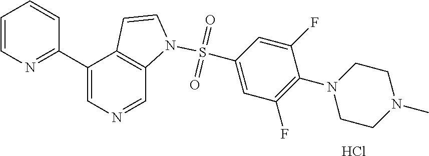 [SMILES: CN1CCN(c2c(F)cc(S(=O)(=O)n3ccc4c(-c5ccccn5)cncc43)cc2F)CC1 ; Cl] | 1-((3,5-difluoro-4-(4-methylpiperazin-1- yl)phenyl)sulfonyl)-4-(pyridin-2-yl)-1H- pyrrolo[2,3-c]pyridine (HCl salt) |

### General Synthesis Methods

Optimum reaction conditions and reaction time may vary depending on the specific reagents used. Unless otherwise indicated, solvents, temperatures, pressures, and other reaction conditions can be freely chosen by one skilled in the art.

The general formula of the intermediates for the synthesis (R₁, R₂, R₃, R₄, and R₅ are defined as above):

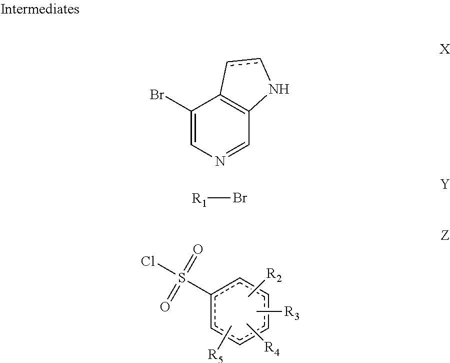

Intermediate X (general structure) for synthesis, bromine derivatives (Intermediates Y) and sulfonyl chloride derivatives (Intermediates Z) are either commercially available or readily prepared by methods known to those skilled in the art.

### Synthesis of Compounds According to the Invention

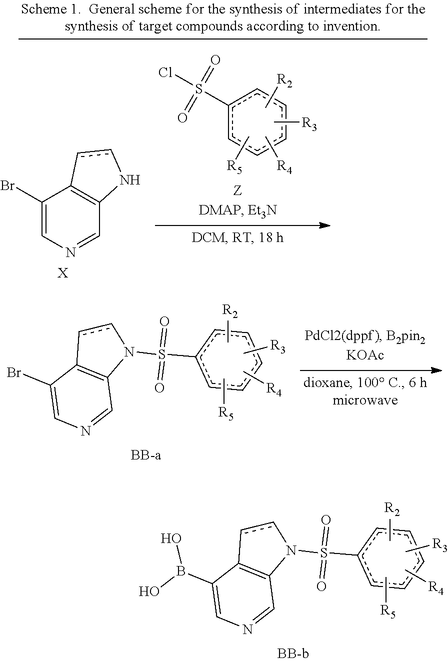

### Preparation of Intermediate BB-a

To a solution of intermediate X (1 equiv.) in DCM (dichloromethane) is added DMAP (4(dimethylamino)pyridine) (0.1 equiv.), triethylamine (2 equiv.) and intermediate Z (1.3 equiv.).

The reaction mixture is stirred at room temperature for 18 hours. The reaction mixture is neutralized with water and extracted with DCM (dichloromethane). The organic phase is washed with brine, then dried over anhydrous sodium sulfate, filtered and concentrated under reduced pressure. The residue is purified by silica gel column chromatography to give intermediate BB-a.

### Obtaining Intermediate BB-b

To a solution of Intermediate BB-a (1 equiv.) in 1,4-dioxane is added B₂pin₂ (bis(pinacolato)diborone) (1.5 equiv.), potassium acetate (3 equiv.) and then degassed in the presence of PdCl₂(dppf) (bis(diphenylphosphino)ferrocene]dichloropalladium(II)) (0.5 equiv.). The reaction mixture is heated at 100° C. under microwave conditions for 6 hours. The reaction mixture is allowed to cool to room temperature and filtered through a pad of celite. Ethyl acetate and water are added to the filtrate. The layers are separated and the aqueous phase is extracted with ethyl acetate. The combined organic layer is washed with brine, dried over anhydrous sodium sulfate, filtered and concentrated under reduced pressure to give intermediate BB-b, which is used in the next step without further purification.

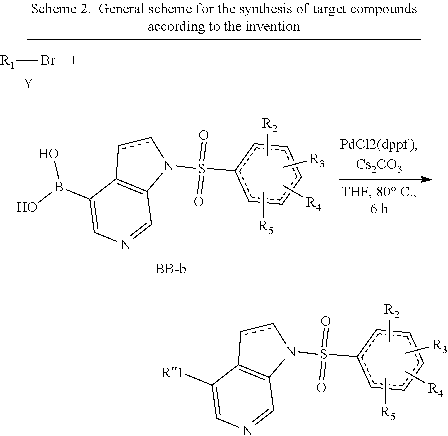

### Preparation of a Compound of the Invention

To a solution of Intermediate BB-b (1 equiv.) in a THF (tetrahydrofuran)/water (10/1) mixture is added intermediate Y (1.2 equiv.), cesium carbonate (2 equiv.) and then degassed in the presence of PdCl₂(dppf) (bis(diphenylphosphino)ferrocene]dichloropalladium(II)) (0.5 equiv.). The reaction mixture is stirred at 80° C. for 6 hours. The reaction mixture is allowed to cool to room temperature and filtered through a pad of celite. Ethyl acetate and water are added to the filtrate. The layers are separated and the aqueous phase is extracted with ethyl acetate. The combined organic layer is washed with brine, dried over anhydrous sodium sulfate, filtered and concentrated under reduced pressure. The residue is purified by silica gel column chromatography.

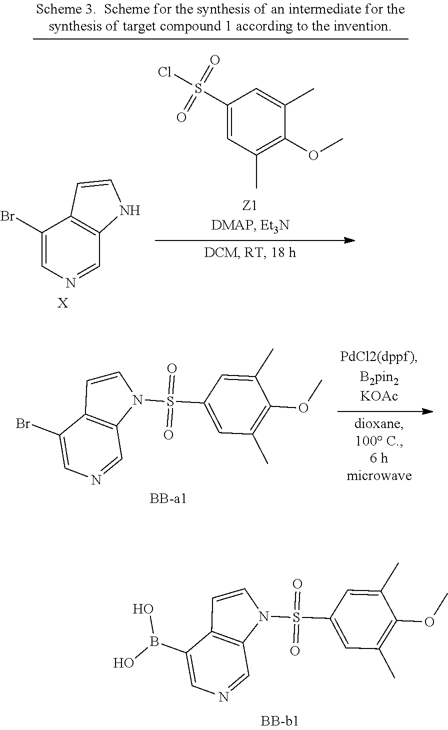

### Preparation of 4-bromo-1-(4-methoxy-3,5-dimethylphenyl)sulfonylpyrrolo[2,3-c]pyridine (BB-a1)

To a solution of 4-bromo-1H-pyrrolo[2,3-c]pyridine (500 mg, 2.54 mmol) in DCM (dichloromethane) (8.0 ml) is added DMAP (4(dimethylamino)pyridine) (31 mg, 0.25 mmol), triethylamine (707 μl, 5.08 mmol) and 4-methoxy-3,5-dimethylbenzenesulfonyl chloride (Intermediate Z1) (774 mg, 3.30 mmol). The reaction mixture is stirred at room temperature for 18 hours. The reaction mixture is neutralized with water and extracted with DCM (dichloromethane). The organic phase is washed with brine, then dried over anhydrous sodium sulfate, filtered and concentrated under reduced pressure. The residue is purified by silica gel column chromatography using 100/0 to 90/10 cyclohexane/ethyl acetate as eluent to give 4-bromo-1-((4-methoxy-3,5-dimethylphenyl)sulfonyl)-1H-pyrrolo[2,3-c]pyridine (BB-a1) (1.05 g, quantitative) as a colorless oil.

### Preparation of [1-(4-methoxy-3,5-dimethylphenyl)sulfonylpyrrolo[2,3-c]pyridin-4-yl]boronic acid (BB-b1)

To a solution of 4-bromo-1-((4-methoxy-3,5-dimethylphenyl)sulfonyl)-1H-pyrrolo[2,3-c]pyridine (BB-a1) (1.05 g, 2.53 mmol) in 1,4-dioxane (15.2 ml) was added B₂pin₂ (bis(pinacolato)diborone) (963 mg, 3.80 mmol), potassium acetate (744 mg, 7.59 mmol) and then degassed in the presence of PdCl₂(dppf) (bis(diphenylphosphino)ferrocene]dichloropalladium(II)) (370 mg, 0.51 mmol). The reaction mixture is heated at 100° C. under microwave conditions for 6 hours. The reaction mixture is allowed to cool to room temperature and filtered through a pad of celite. Ethyl acetate and water are added to the filtrate. The layers are separated and the aqueous phase is extracted with ethyl acetate. The combined organic layer was washed with brine, dried over anhydrous sodium sulfate, filtered and concentrated under reduced pressure to give (1-((4-methoxy-3,5-dimethylphenyl)sulfonyl)-1H-pyrrolo[2,3-c]pyridin-4-yl)boronic acid (BB-b1) (1.99 g, 99% yield) as a brown oil, which is used in the next step without further purification.

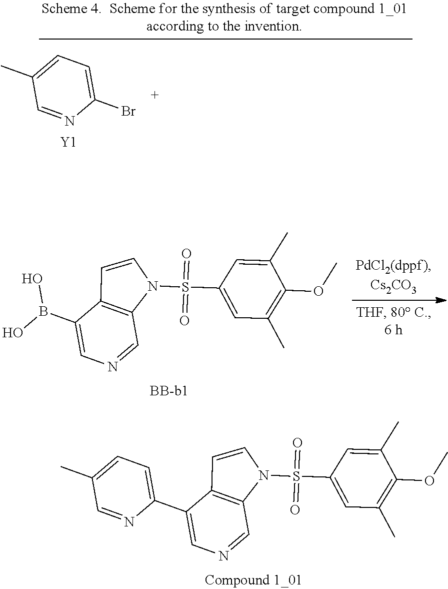

### Preparation of 1-(4-methoxy-3,5-dimethylphenyl)sulfonyl-4-(5-methyl-2-pyridyl)pyrrolo[2,3-c]pyridine (Compound 1_01)

To a solution of (1-((4-methoxy-3,5-dimethylphenyl)sulfonyl)-1H-pyrrolo[2,3-c]pyridin-4-yl)boronic acid (BB-b1) (200 mg, 45% wt., 0.25 mmol) in THF (tetrahydrofuran) (1.1 ml) and water (0.11 ml) was added 2-bromo-5-methylpyridine (53.7 mg, 0.31 mmol), cesium carbonate (163 mg, 0.50 mmol) and then degassed in the presence of PdCl₂(dppf) (bis(diphenylphosphino)ferrocene]dichloropalladium(II)) (9.14 mg, 0.13 mmol). The reaction mixture was stirred at 80° C. for 6 hours. The reaction mixture was allowed to cool to room temperature and filtered through a pad of celite. Ethyl acetate and water were added to the filtrate. The layers were separated and the aqueous phase was extracted with ethyl acetate. The combined organic layer was washed with brine, dried over anhydrous sodium sulfate, filtered and concentrated under reduced pressure. The residue was purified by chromatography on a silica gel column using 100/0 to 60/40 cyclohexane/ethyl acetate as eluent. The resulting solid was triturated in diethyl ether, filtered and dried to give 1-((4-methoxy-3,5-dimethylphenyl)sulfonyl)-4-(5-methylpyridin-2-yl)-1H-pyrrolo[2,3-c]pyridine (Compound 1_01) (10.4 mg, 9.7% yield) as a white solid.

Compounds 1_02-1_33 are prepared similarly to the procedure above, but using the characteristic bromine derivative of intermediate Y and the sulfonyl chloride derivative of intermediate Z.

### General Approach for the Synthesis of Compounds 2_01-4_01 According to the Invention

The general formula of the intermediates for the synthesis (R₁, R₂ are defined as above):

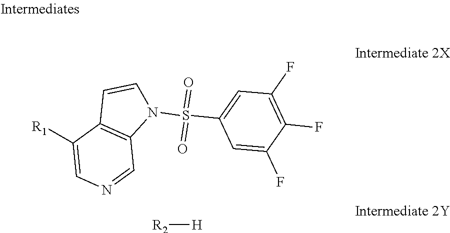

Intermediate 2X (general structure) for synthesis, and Intermediates 2Y are either commercially available or readily prepared by methods known to those skilled in the art.

### General Scheme for the Synthesis of Compounds 2 01-4 01 According to the Invention

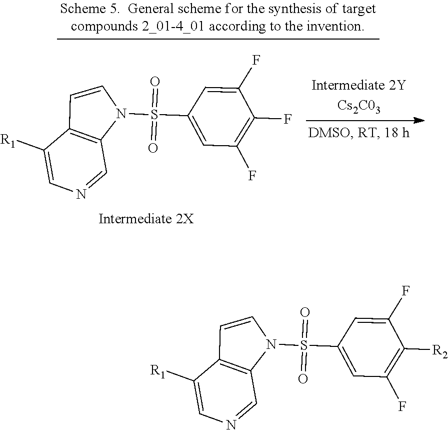

To a mixture of Intermediate 2Y (1 equiv.) in DMSO is added Cs₂CO₃ (502 mg, 2.67 equiv.). After 15 minutes at room temperature, Intermediate 2X (0.67 eq.) is added to the reaction mixture, and the reaction mixture is stirred at room temperature for 18 hours. The mixture is purified directly by preparative HPLC to give the compound of the invention.

### Obtaining Some Specific Compounds According to the Invention

### Preparation of 1-((3,5-difluoro-4-((1R,4R)-5-methyl-2,5-diazabicyclo[2.2.1]heptan-2-yl)phenyl)sulfonyl)-4-(pyridine)-2-yl)-1H-pyrrolo[2,3-c]pyridine (compound 2_02)

To a mixture of 1-cyclopropylmethylpiperazine (85.9 μl, 578 μmol) in DMSO (2.2 ml) was added Cs₂CO₃ (502 mg, 1.54 mmol). After 15 minutes at room temperature, 4-(pyridin-2-yl)-1-((3,4,5-trifluorophenyl)sulfonyl)-1H-pyrrolo[2,3-c]pyridine (150 mg, 385 μmol) was added and the reaction mixture was stirred at room temperature for 18 hours. The mixture was purified directly by preparative HPLC to give 1-((4-(4-(cyclopropylmethyl)piperazin-1-yl)-3,5-difluorophenyl)sulfonyl)-4-(pyridin-2-yl)-1H-pyrrolo[2,3-c]pyridine (54 mg, 26% yield) as a white solid.

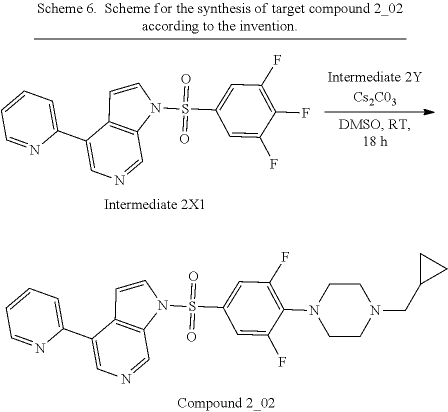 [SMILES: O=S(=O)(c1cc(F)c(F)c(F)c1)n1ccc2c(-c3ccccn3)cncc21 ; O=S(=O)(c1cc(F)c(N2CCN(CC3CC3)CC2)c(F)c1)n1ccc2c(-c3ccccn3)cncc21]

### Preparation of 1-((4-(1,4-diazabicyclo[3.2.1]octan-4-yl)-3,5-difluorophenyl)sulfonyl)-4-(pyridin-2-yl)-1H-pyrrolo[2,3-c]pyridine (compound 3_20)

To a mixture of 1,4-diazabicyclo[3.2.1]octane dihydrochloride (107 mg, 578 μmol) in DMSO (2.2 ml) was added Cs₂CO₃ (502 mg, 1.54 mmol). After 15 minutes at room temperature, 4-(pyridin-2-yl)-1-((3,4,5-trifluorophenyl)sulfonyl)-1H-pyrrolo[2,3-c]pyridine (intermediate 2, 150 mg, 385 μmol) was added and the reaction mixture was stirred at room temperature for 3 hours. The mixture was purified directly by preparative HPLC column chromatography to give 1-((4-(1,4-diazabicyclo[3.2.1]octan-4-yl)-3,5-difluorophenyl)sulfonyl)-4-(pyridin-2-yl)-1H-pyrrolo[2,3-c]pyridine (13 mg, 6.7% yield) as a white solid.

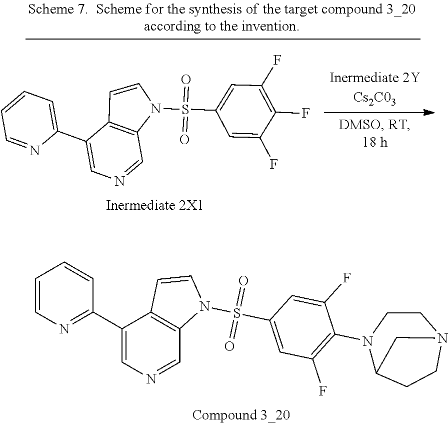 [SMILES: O=S(=O)(c1cc(F)c(F)c(F)c1)n1ccc2c(-c3ccccn3)cncc21 ; O=S(=O)(c1cc(F)c(N2CCN3CCC2C3)c(F)c1)n1ccc2c(-c3ccccn3)cncc21]

Compounds 2_01, 2_03-3_19 and 3_21-4_01 are obtained similarly to the procedure above, but using characteristic intermediates.

### Experimental Analysis

LC-MS analysis of compounds according to the invention.

Analytical HPLC was conducted on a X-Select CSH C18 XP column (2.5 μm 30×4.6 mm id) eluting with 0.1% formic acid in water (solvent A) and 0.1% formic acid in acetonitrile (solvent B), using the following elution gradient 0-3 minutes: 5% to 100% B, 3-4 minutes 100% B, at a flow rate of 1.8 mL/minute at 40° C. The mass spectra (MS) were recorded on a Waters ZQ mass spectrometer (scan 200-900 uma) using electrospray positive ionization [ES+ to give [M+H+ molecular ions] or electrospray negative ionization [ES− to give [M−H]− molecular ions] modes with a 20 V cone voltage.

### Example 1

Compound 1_01: 1-(4-methoxy-3,5-dimethylphenyl)sulfonyl-4-(5-methyl-2-pyridyl)pyrrolo[2,3-c]pyridine; MW=407.49; Purity (by HPLC) >95%, MS (ESI+), m/z 408.4 [M+H]+.

### Example 2

Compound 1_02: 1-((4-Methoxy-3,5-dimethylphenyl)sulfonyl)-4-(5-methylpyrazin-2-yl)-1H-pyrrolo[2,3-c]pyridine; MW=408.47; Purity (by HPLC) >95%, MS (ESI+), m/z 409.4 [M+H]+.

### Example 3

Compound 1_03: 1-((4-methoxy-3,5-dimethylphenyl)sulfonyl)-4-(5-methylpyrimidin-2-yl)-1H-pyrrolo[2,3-c]pyridine; MW=408.47; Purity (by HPLC) >95%, MS (ESI+), m/z 409.4 [M+H]+.

### Example 4

Compound 1_04: 1-(4-methoxy-3,5-dimethylphenyl)sulfonyl-4-(6-methylpyridazin-3-yl)pyrrolo[2,3-c]pyridine; MW=409.3; Purity (by HPLC) >95%, MS (ESI+), m/z 395.2 [M+H]+.

### Example 5

Compound 1_05: 1-(4-methoxy-3,5-dimethylphenyl)sulfonyl-4-(5-methylsulfonyl-2-pyridyl)pyrrolo[2,3-c]pyridine; MW=471.55; Purity (by HPLC) >95%, MS (ESI+), m/z 472.1 [M+H]+.

### Example 6

Compound 1_06: 1-((3,5-difluoro-4-isopropoxyphenyl)sulfonyl)-4-(pyridin-2-yl)-1H-pyrrolo[2,3-c]pyridine; MW=429.44; Purity (by HPLC) >95%, MS (ESI+), m/z 430.1 [M+H]+.

### Example 7

Compound 1_07: 1-((4-(cyclopropylmethoxy)-3,5-difluorophenyl)sulfonyl)-4-(pyridin-2-yl)-1H-pyrrolo[2,3-c]pyridine; MW=441.45; Purity (by HPLC) >95%, MS (ESI+), m/z 442.3 [M+H]+.

### Example 8

Compound 1_08: 1-((4-(2,2-difluoroethoxy)-3,5-difluorophenyl)sulfonyl)-4-(pyridin-2-yl)-1H-pyrrolo[2,3-c]pyridine; MW=451.4; Purity (by HPLC) >95%, MS (ESI+), m/z 452.0 [M+H]+.

### Example 9

Compound 1_09: 2,6-difluoro-N,N-dimethyl-4-((4-(pyridin-2-yl)-1H-pyrrolo[2,3-c]pyridin-1-yl)sulfonyl)aniline; MW=414.43; Purity (by HPLC) >95%, MS (ESI+), m/z 415.1 [M+H]+.

### Example 10

Compound 1_10: 4-(2,6-difluoro-4-((4-(pyridin-2-yl)-1H-pyrrolo[2,3-c]pyridin-1-yl)sulfonyl)phenyl)morpholine; MW=456.47; Purity (by HPLC) >95%, MS (ESI+), m/z 457.1 [M+H]+.

### Example 11

Compound 1_11: 2,6-difluoro-4-((4-(pyridin-2-yl)-1H-pyrrolo[2,3-c]pyridin-1-yl)sulfonyl)aniline; MW=386.38; Purity (by HPLC) >95%, MS (ESI+), m/z 387.3 [M+H]+.

### Example 12

Compound 1_12: 1-((3,5-difluoro-4-(4-methylpiperazin-1-yl)phenyl)sulfonyl)-4-(pyridin-2-yl)-1H-pyrrolo[2,3-c]pyridine; MW=469.51; Purity (by HPLC) >95%, MS (ESI+), m/z 470.2 [M+H]+.

### Example 13

Compound 1_13: N-cyclopropyl-2,6-difluoro-4-((4-(pyridin-2-yl)-1H-pyrrolo[2,3-c]pyridin-1-yl)sulfonyl)aniline; MW=426.44; Purity (by HPLC) >95%, MS (ESI+), m/z 427.4 [M+H]+.

### Example 14

Compound 1_14: N-cyclobutyl-2,6-difluoro-N-methyl-4-((4-(pyridin-2-yl)-1H-pyrrolo[2,3-c]pyridin-1-yl)sulfonyl)aniline; MW=454.5; Purity (by HPLC) >95%, MS (ESI+), m/z 455.3 [M+H]+.

### Example 15

Compound 1_15: 2,6-difluoro-N,N-dimethyl-4-((4-(pyridin-2-yl)-2,3-dihydro-1H-pyrrolo[2,3-c]pyridin-1-yl)sulfonyl)aniline; MW=416.45; Purity (by HPLC) >95%, MS (ESI+), m/z 417.4 [M+H]+.

### Example 16

Compound 1_16: 2,6-difluoro-4-((4-(pyridin-2-yl)-2,3-dehydro-H-pyrrolo[2,3-c]pyridin-1-yl)sulfonyl)phenol; MW=389.38; Purity (by HPLC) >95%, MS (ESI+), m/z *** [M+H]+.

### Example 17

Compound 1_17: 1-((3,5-difluoro-4-(4-methylpiperazin-1-yl)phenyl)sulfonyl)-4-(pyridin-2-yl)-2,3-dihydro-1H-pyrrolo[2,3-c]pyridine; MW=471.53; Purity (by HPLC) >95%, MS (ESI+), m/z 472.4 [M+H]+.

### Example 18

Compound 1_18: 1-((3,5-difluoro-4-methoxyphenyl)sulfonyl)-4-(pyridin-2-yl)-1H-pyrrolo[2,3-c]pyridine; MW=401.39; Purity (by HPLC) >95%, MS (ESI+), m/z ** [M+H]+.

### Example 19

Compound 1_19: 1-((3,5-difluoro-4-(3-methylazetidin-1-yl)phenyl)sulfonyl)-4-(pyridin-2-yl)-1H-pyrrolo[2,3-c]pyridine; MW=440.47; Purity (by HPLC) >95%, MS (ESI+), m/z 441.1 [M+H]+.

### Example 20

Compound 1_20: N-(2,6-difluoro-4-((4-(pyridin-2-yl)-1H-pyrrolo[2,3-c]pyridin-1-yl)sulfonyl)phenyl)-N,4-dimethylthiazol-2-amine; MW=497.54; Purity (by HPLC) >95%, MS (ESI+), m/z 498.3 [M+H]+.

### Example 21

Compound 1_21: N-(2,6-difluoro-4-((4-(pyridin-2-yl)-1H-pyrrolo[2,3-c]pyridin-1-yl)sulfonyl)phenyl)-1-methylpiperidin-4-amine; MW=483.54; Purity (by HPLC) >95%, MS (ESI+), m/z 484.1 [M+H]+.

### Example 22

Compound 1_22: N1-(2,6-difluoro-4-((4-(pyridin-2-yl)-1H-pyrrolo[2,3-c]pyridin-1-yl)sulfonyl)phenyl)-N2,N2-dimethylethane-1,2-diamine; MW=457.5; Purity (by HPLC) >95%, MS (ESI+), m/z 458.4 [M+H]+.

### Example 23

Compound 1_23: 4-(2,6-difluoro-4-((4-(pyridin-2-yl)-1H-pyrrolo[2,3-c]pyridin-1-yl)sulfonyl)phenyl)thiomorpholine; MW=472.53; Purity (by HPLC) >95%, MS (ESI+), m/z 473.3 [M+H]+.

### Example 24

Compound 1_24: 1-((3,5-difluoro-4-(5-methylhexahydropyrrolo[3,4-c]pyrrol-2(1H)-yl)phenyl)sulfonyl)-4-(pyridin-2-yl)-1H-pyrrolo [2,3-s]pyridine; MW=495.55; Purity (by HPLC) >95%, MS (ESI+), m/z 496.4 [M+H]+.

### Example 25

Compound 1_25: 1-((3,5-difluoro-4-(5-methyloctahydro-2H-pyrrolo[3,4-c]pyridin-2-yl)phenyl)sulfonyl)-4-(pyridin-2-yl)-1H-pyrrolo[2,3-c]pyridine; MW=495.55; Purity (by HPLC) >95%, MS (ESI+), m/z 496.2 [M+H]+.

### Example 26

Compound 1_26: 2-(2,6-difluoro-4-((4-(pyridin-2-yl)-1H-pyrrolo[2,3-c]pyridin-1-yl)sulfonyl)phenyl)-2,7-diazaspiro[4.4]nonane; MW=509.58; Purity (by HPLC) >95%, MS (ESI+), m/z 510.2 [M+H]+.

### Example 27

Compound 1_27: 2-(1-((3,5-difluoro-4-(4-methylpiperazin-1-yl)phenyl)sulfonyl)-1H-pyrrolo[2,3-c]pyridin-4-yl)oxazol-4-carbonitrile; MW=484.48; Purity (by HPLC) >95%, MS (ESI+), m/z 485.4 [M+H]+.

### Example 28

Compound 1_28: 5-(1-((3,5-difluoro-4-(4-methylpiperazin-1-yl)phenyl)sulfonyl)-1H-pyrrolo[2,3-c]pyridin-4-yl)picolinonitrile; MW=494.52; Purity (by HPLC) >95%, MS (ESI+), m/z 495.4 [M+H]+.

### Example 29

Compound 1_29: 1-((3,5-difluoro-4-(4-methylpiperazin-1-yl)phenyl)sulfonyl)-4-(5-fluoropyrimidin-2-yl)-1H-pyrrolo[2,3-c]pyridine; MW=488.49; Purity (by HPLC) >95%, MS (ESI+), m/z 489.1 [M+H]+.

### Example 30

Compound 1_30: 55-(1-((3,5-difluoro-4-(4-methylpiperazin-1-yl)phenyl)sulfonyl)-1H-pyrrolo[2,3-c]pyridin-4-yl)-N-methylpyrazin-2-amine; MW=499.54; Purity (by HPLC) >95%, MS (ESI+), m/z 500.2 [M+H]+.

### Example 31

Compound 1_31: 4-(5-cyclopropylpyridin-2-yl)-1-((3,5-difluoro-4-(4-methylpiperazin-1-yl)phenyl)sulfonyl)-1H-pyrrolo[2,3-c]pyridine; MW=509.58; Purity (by HPLC) >95%, MS (ESI+), m/z 510.4 [M+H]+.

### Example 32

Compound 1_32: 2,6-difluoro-4-((4-(5-methylpyrazin-2-yl)-1H-pyrrolo[2,3-c]pyridin-1-yl)sulfonyl)aniline; MW=401.39; Purity (by HPLC) >95%, MS (ESI+), m/z 402.1 [M+H]+.

### Example 33

Compound 1_33: 2,6-difluoro-N,N-dimethyl-4-((4-(5-methylpyridin-2-yl)-1H-pyrrolo[2,3-c]pyridin-1-yl)sulfonyl)aniline; MW=428.46; Purity (by HPLC) >95%, MS (ESI+), m/z 429.1 [M+H]+.

### Example 34

Compound 2_01: 4-(2,6-difluoro-4-((4-(pyridin-2-yl)-1H-pyrrolo[2,3-c]pyridin-1-yl)sulfonyl)phenyl)-1-methylpiperazin-2-one; MW=483.49; Purity (by HPLC) >95%, MS (ESI+), m/z 484.1 [M+H]+.

### Example 35

Compound 2_02: 1-((4-(4-(cyclopropylmethyl)piperazin-1-yl)-3,5-difluorophenyl)sulfonyl)-4-(pyridin-2-yl)-1H-pyrrolo[2,3-c]pyridine; MW=509.58; Purity (by HPLC) >95%, MS (ESI+), m/z 510.4 [M+H]+.

### Example 36

Compound 2_03: N-(tert-butyl)-5-(1-((3,5-difluoro-4-(5-methylhexahydropyrrolo[3,4-c]pyrrol-2(1H)-yl)phenyl)sulfonyl)-1H-pyrrolo[2,3-c]pyridin-4-yl)picolinamide; MW=594.68; Purity (by HPLC) >95%, MS (ESI+), m/z 595.5 [M+H]+.

### Example 37

Compound 3_01: 1-((3,5-difluoro-4-(piperazin-1-yl)phenyl)sulfonyl)-4-(pyridin-2-yl)-1H-pyrrolo[2,3-c]pyridine; MW=455.48; Purity (by HPLC) >95%, MS (ESI+), m/z 456.1 [M+H]+.

### Example 38

Compound 3_02: 6-(1-((3,5-difluoro-4-(4-methylpiperazin-1-yl)phenyl)sulfonyl)-1H-pyrrolo[2,3-c]pyridin-4-yl)-N-methylpyridin-3-amine; MW=498.55; Purity (by HPLC) >95%, MS (ESI+), m/z 499.4 [M+H]+.

### Example 39

Compound 3_03: 5-(1-((3,5-difluoro-4-(4-methylpiperazin-1-yl)phenyl)sulfonyl)-1H-pyrrolo[2,3-c]pyridin-4-yl)-N-methylpyridin-2-amine; MW=498.55; Purity (by HPLC) >95%, MS (ESI+), m/z 499.1 [M+H]+.

### Example 40

Compound 3_04: 6-(1-((3,5-difluoro-4-(4-methylpiperazin-1-yl)phenyl)sulfonyl)-1H-pyrrolo[2,3-c]pyridin-4-yl)-N,N-dimethylpyridin-3-amine; MW=512.58; Purity (by HPLC) >95%, MS (ESI+), m/z 513.4 [M+H]+.

### Example 41

Compound 3_05: 5-(1-((3,5-difluoro-4-(4-methylpiperazin-1-yl)phenyl)sulfonyl)-1H-pyrrolo[2,3-c]pyridin-4-yl)pyrazine-2-carbonitrile; MW=495.51; Purity (by HPLC) >95%, MS (ESI+), m/z 496.3 [M+H]+.

### Example 42

Compound 3_06: 6-(1-((3,5-difluoro-4-(4-methylpiperazin-1-yl)phenyl)sulfonyl)-1H-pyrrolo[2,3-c]pyridin-4-yl)nicotinonitrile; MW=494.52; Purity (by HPLC) >95%, MS (ESI+), m/z 495.3 [M+H]+.

### Example 43

Compound 3_07: 6-(1-((3,5-difluoro-4-(4-methylpiperazin-1-yl)phenyl)sulfonyl)-1H-pyrrolo[2,3-c]pyridin-4-yl)-N-methylpyridazin-3-amine; MW=499.54; Purity (by HPLC) >95%, MS (ESI+), m/z 500.4 [M+H]+.

### Example 44

Compound 3_08: 2-(1-((3,5-difluoro-4-(4-methylpiperazin-1-yl)phenyl)sulfonyl)-1H-pyrrolo[2,3-c]pyridin-4-yl)-N-methylpyrimidin-5-amine; MW=499.54; Purity (by HPLC) >95%, MS (ESI+), m/z 500.4 [M+H]+.

### Example 45

Compound 3_09: 5-(1-((3,5-difluoro-4-(4-methylpiperazin-1-yl)phenyl)sulfonyl)-1H-pyrrolo[2,3-c]pyridin-4-yl)pyrimidine-2-carbonitrile; MW=495.51; Purity (by HPLC) >95%, MS (ESI+), m/z 496.3 [M+H]+.

### Example 46

Compound 3_10: 6-(1-((3,5-difluoro-4-(4-methylpiperazin-1-yl)phenyl)sulfonyl)-1H-pyrrolo[2,3-c]pyridin-4-yl)pyridazine-3-carbonitrile; MW=495.51; Purity (by HPLC) >95%, MS (ESI+), m/z 496.3 [M+H]+.

### Example 47

Compound 3_11: 1-((3,5-difluoro-4-(4-isopropylpiperazin-1-yl)phenyl)sulfonyl)-4-(pyridin-2-yl)-1H-pyrrolo[2,3-c]pyridine; MW=497.56; Purity (by HPLC) >95%, MS (ESI+), m/z 498.4 [M+H]+.

### Example 48

Compound 3_12: 1-((4-(4-cyclopropylpiperazin-1-yl)-3,5-difluorophenyl)sulfonyl)-4-(pyridin-2-yl)-1H-pyrrolo[2,3-c]pyridine; MW=495.55; Purity (by HPLC) >95%, MS (ESI+), m/z 496.4 [M+H]+.

### Example 49

Compound 3_13: 1-((3,5-difluoro-4-(4-methyl-1,4-diazepan-1-yl)phenyl)sulfonyl)-4-(pyridin-2-yl)-1H-pyrrolo[2,3-c]pyridine; MW=483.54; Purity (by HPLC) >95%, MS (ESI+), m/z 484.1 [M+H]+.

### Example 50

Compound 3_14: 1-((4-(1,4-diazepan-1-yl)-3,5-difluorophenyl)sulfonyl)-4-(pyridin-2-yl)-1H-pyrrolo[2,3-c]pyridine; MW=469.51; Purity (by HPLC) >95%, MS (ESI+), m/z 470.1 [M+H]+.

### Example 51

Compound 3_15: 1-(2,6-difluoro-4-((4-(pyridin-2-yl)-1H-pyrrolo[2,3-c]pyridin-1-yl)sulfonyl)phenyl)piperidin-4-amine; MW=469.51; Purity (by HPLC) >95%, MS (ESI+), m/z 470.4 [M+H]+.

### Example 52

Compound 3_16: 1-((3,5-difluoro-4-(6-methyl-2,6-diazaspiro[3.3]heptan-2-yl)phenyl)sulfonyl)-4-(pyridin-2-yl)-1H-pyrrolo[2,3-c]pyridine; MW=481.52; Purity (by HPLC) >95%, MS (ESI+), m/z 482.4 [M+H]+.

### Example 53

Compound 3_17: 1-((3,5-difluoro-4-(5-methyl-2,5-diazabicyclo[2.2.1]heptan-2-yl)phenyl)sulfonyl)-4-(pyridin-2-yl)-1H-pyrrolo[2,3-c]pyridine; MW=481.52; Purity (by HPLC) >95%, MS (ESI+), m/z 482.2 [M+H]+.

### Example 54

Compound 3_18: 6-(2,6-difluoro-4-((4-(pyridin-2-yl)-1H-pyrrolo[2,3-c]pyridin-1-yl)sulfonyl)phenyl)-3-methyl-3,6-diazabicyclo[3.1.1]heptane; MW=481.52; Purity (by HPLC) >95%, MS (ESI+), m/z 482.3 [M+H]+.

### Example 55

Compound 3_19: 1-((3,5-difluoro-4-(3-methyl-3,8-diazabicyclo[3.2.1]octan-8-yl)phenyl)sulfonyl)-4-(pyridin-2-yl)-1H-pyrrolo[2,3-c]pyridine; MW=495.55; Purity (by HPLC) >95%, MS (ESI+), m/z 496.4 [M+H]+.

### Example 56

Compound 3_20: 1-((4-(1,4-diazabicyclo[3.2.1]octan-4-yl)-3,5-difluorophenyl)sulfonyl)-4-(pyridin-2-yl)-1H-pyrrolo[2,3-c]pyridine; MW=481.52; Purity (by HPLC) >95%, MS (ESI+), m/z 482.3 [M+H]+.

### Example 57

Compound 3_21: 1-((3,5-difluoro-4-(2,6-diazaspiro[3.3]heptan-2-yl)phenyl)sulfonyl)-4-(pyridin-2-yl)-1H-pyrrolo[2,3-c]pyridine; MW=467.49; Purity (by HPLC) >95%, MS (ESI+), m/z 468.4 [M+H]+.

### Example 58

Compound 3_22: 5-(1-((3,5-difluoro-4-(5-methylhexahydropyrrolo[3,4-c]pyrrol-2(1H)-yl)phenyl)sulfonyl)-1H-pyrrolo[2,3-c]pyridin-4-yl)-N-methylpyrazin-2-amine; MW=525.58; Purity (by HPLC) >95%, MS (ESI+), m/z 526.4 [M+H]+.

### Example 59

Compound 3_23: 5-(1-((3,5-difluoro-4-(5-methylhexahydropyrrolo[3,4-c]pyrrol-2(1H)-yl)phenyl)sulfonyl)-1H-pyrrolo[2,3-c]pyridin-4-yl)pyrazine-2-carbonitrile; MW=521.55; Purity (by HPLC) >95%, MS (ESI+), m/z 522.4 [M+H]+.

### Example 60

Compound 3_24: 5-(1-((3,5-difluoro-4-(5-methylhexahydropyrrolo[3,4-c]pyrrol-2(1H)-yl)phenyl)sulfonyl)-1H-pyrrolo[2,3-c]pyridin-4-yl)picolinonitrile; MW=520.56; Purity (by HPLC) >95%, MS (ESI+), m/z 521.4 [M+H]+.

### Example 61

Compound 3_25: 1-((4-(2,5-diazabicyclo[2.2.1]heptan-2-yl)-3,5-difluorophenyl)sulfonyl)-4-(pyridin-2-yl)-1H-pyrrolo[2,3-c]pyridine (HCl salt); MW=467.49; Purity (by HPLC) >95%, MS (ESI+), m/z 468.2 [M+H]+.

### Example 62

Compound 3_26: 1-((4-(3,8-diazabicyclo[3.2.1]octan-8-yl)-3,5-difluorophenyl)sulfonyl)-4-(pyridin-2-yl)-1H-pyrrolo[2,3-c]pyridine; MW=481.52; Purity (by HPLC) >95%, MS (ESI+), m/z 482.2 [M+H]+.

### Example 63

Compound 3_27: 1-((3,5-difluoro-4-(piperazin-1-yl)phenyl)sulfonyl)-4-(pyridin-2-yl)-1H-pyrrolo[2,3-c]pyridine (HCl salt); MW=455.48; Purity (by HPLC) >95%, MS (ESI+), m/z 456.2 [M+H]+.

### Example 64

Compound 3_28: 5-(1-((3,5-difluoro-4-(hexahydropyrrolo[3,4-c]pyrrol-2(1H)-yl)phenyl)sulfonyl)-1H-pyrrolo[2,3-c]pyridin-4-yl)picolinonitrile; MW=506.53; Purity (by HPLC) >95%, MS (ESI+), m/z 507.4 [M+H]+.

### Example 65

Compound 4_01: 1-((3,5-difluoro-4-(4-methylpiperazin-1-yl)phenyl)sulfonyl)-4-(pyridin-2-yl)-1H-pyrrolo[2,3-c]pyridine (HCl salt); MW=469.51; Purity (by HPLC) >95%, MS (ESI+), m/z 470.2 [M+H]+.

### Characteristics of the Biological Activity of the Compounds of the Invention

The authors of the present invention investigated the activity of the compounds according to the invention as inhibitors of CYP11 B2 when analyzed in a reconstructed system containing 0.5 μm recombinant CYP1 1B2, 0.3 μm human adrenodoxin reductase, 2 μm human adrenodoxin, 100 μm corticosterone as a control reaction to detect the formation of a product with using an HPLC system with a UV detector. The test compound is added to the reaction mixture to a final concentration of ≤2 μM. The concentrated protein mixture was diluted with buffer to a final volume of 0.5 ml, pre-incubated for 10 min at 37° C., followed by the addition of the substrate. Reactions are initiated by adding NADPH (nicotinamide adenine dinucleotide phosphate) to a final concentration of 0.25 mM with a regenerating system (glucose-6-phosphate and glucose-6-phosphate dehydrogenase) in 25 mM potassium phosphate buffer, pH 7.4, containing 0.1 mM DTT (dithiothreitol), 0.1% Tween-20.4 mM MgCl₂ and incubated at 37° C. After 30 minutes of incubation, the reaction is stopped by adding 5 ml of dichloromethane. The organic phase is isolated by centrifugation, evaporation, dissolution in ethanol and transferred to a vial for HPLC studies. A C18 Luna 100 Å 250×4, 6 mm column was used on an Agilent Technologies 1200 series instrument (USA) with ethanol as the mobile phase.

Activity (a) is calculated as the amount of product in nmol per minute per 1 nmol of CYP.

$$a = \frac{k \times {A}_{p} \times {c}_{s}}{( {A}_{p} + {A}_{s} ) \times {c}_{e} \times t} ,$$

where k is the correction factor (k=1 is used as the molar extinction coefficients of steroid substrates and products similar at a detection wavelength of 254 nm), Aₛ is the peak area of the substrate, cₛ is the concentration of the substrate, Aₚ is the peak area of the product, cₑ is the concentration of CYP (cytochrome P450), t is the reaction time.

The level of inhibition of CYP11 B2 activity is expressed as a percentage, where 0% is defined as no inhibition in reactions without test compound and 100% is absolute inhibition by test compound. Compounds of all embodiments of the invention are suitable for inhibiting CYP11 B2 activity, and a preferred subgroup includes compounds with inhibition of CYP11 B2 activity in the range of 1-100% (+). Within this subgroup, there is another subgroup of more preferred compounds with inhibition of CYP11B2 activity in the range of 20-100% (++). In the latter, there is another subgroup of more preferred compounds with inhibition of CYP11 B2 activity in the range of 35-100% for compounds 2.1-2.4 and 3.1-3.6 (+++) or 50-100% (+++) for compounds 1.1-1.18. See Table 2. The connection code numbers correspond to the earlier codes given in Table 1.

TABLE 2

| Compound | Level of CYP11B2 |
|---|---|
| No | activity inhibition |
| 1_01 | +++ |
| 1_02 | +++ |
| 1_03 | +++ |
| 1_04 | +++ |
| 1_05 | ++ |
| 1_06 | +++ |
| 1_07 | +++ |
| 1_08 | +++ |
| 1_09 | +++ |
| 1_10 | +++ |
| 1_11 | +++ |
| 1_12 | +++ |
| 1_13 | +++ |
| 1_14 | +++ |
| 1_15 | ++ |
| 1_16 | +++ |
| 1_17 | +++ |
| 1_18 | +++ |
| 1_19 | +++ |
| 1_20 | +++ |
| 1_21 | +++ |
| 1_22 | +++ |
| 1_23 | +++ |
| 1_24 | ++ |
| 1_25 | +++ |
| 1_26 | +++ |
| 1_27 | +++ |
| 1_28 | +++ |
| 1_29 | +++ |
| 1_30 | + |
| 1_31 | +++ |
| 1_32 | +++ |
| 1_33 | +++ |
| 2_01 | +++ |
| 2_02 | +++ |
| 2_03 | +++ |
| 3_01 | ++ |
| 3_02 | +++ |
| 3_03 | ++ |
| 3_04 | +++ |
| 3_05 | +++ |
| 3_06 | +++ |
| 3_07 | +++ |
| 3_08 | ++ |
| 3_09 | +++ |
| 3_10 | +++ |
| 3_11 | +++ |
| 3_12 | ++ |
| 3_13 | +++ |
| 3_14 | +++ |
| 3_15 | +++ |
| 3_16 | +++ |
| 3_17 | +++ |
| 3_18 | +++ |
| 3_19 | +++ |
| 3_20 | +++ |
| 3_21 | ++ |
| 3_22 | +++ |
| 3_23 | +++ |
| 3_24 | +++ |
| 3_25 | +++ |
| 3_26 | +++ |
| 3_27 | ++ |
| 3_28 | +++ |
| 4_01 | +++ |

### Pharmaceutical Compositions

The invention also relates to pharmaceutical compositions which contain a compound of general formula I, Ia, IIa, Ib, IIb, Ic, Id, IId, Ie, If, IIId (or a pro-drug, pharmaceutically acceptable salt, or other pharmaceutically acceptable derivative) and one or more pharmaceutically acceptable carriers, adjuvants, diluents, and/or excipients, such, which can be introduced into the patient's body together with the compound that is the essence of this invention, and which do not destroy the pharmacological activity of this compound, and are non-toxic when administered in doses sufficient to deliver a therapeutic amount of the compound.

The pharmaceutical compositions of this invention contain the compounds of this invention together with pharmaceutically acceptable carriers, which may include any solvents, diluents, dispersions or suspensions, surfactants, isotonic agents, thickeners and emulsifiers, preservatives, binders, lubricants, etc. suitable for the particular dosage form. Materials that can serve as pharmaceutically acceptable carriers include, but are not limited to, mono- and oligosaccharides, as well as their derivatives; gelatin; talc; excipients such as cocoa butter and suppository wax; oils such as peanut, cottonseed, sesame, olive, corn and soybean oils and others; glycols such as propylene glycol; esters such as ethyl oleate and ethyl laurate; agar; buffer substances such as magnesium hydroxide and aluminum hydroxide; alginic acid; depyrogenated water; isotonic solution, Ringer's solution; alcohol and phosphate buffer solutions. Other non-toxic compatible lubricants, such as sodium lauryl sulfate and magnesium stearate, as well as colorants, release agents, filming agents, sweeteners, flavors and fragrances, preservatives and antioxidants may also be included in the composition.

The subject of the present invention are also dosage forms—a class of pharmaceutical compositions, the structure of which is optimized for a certain method of administration to the body in a therapeutically effective dose, for example, for administration to the body intravenously, intramuscularly, orally, subcutaneously, intraocularly, inhalation, intranasally and sublingually, in recommended dosages.

Dosage forms of this invention may contain structures obtained by liposomal methods, microencapsulation methods, methods for obtaining nanoforms of the drug, or other methods known in the pharmaceutical art.

When preparing a composition, for example in the form of a tablet, the active principle is mixed with one or more pharmaceutical excipients, such as gelatin, starch, lactose, magnesium stearate, talc, silicon dioxide, Arabic gum, mannitol, microcrystalline cellulose, hypromellose or similar compounds.

Tablets may be coated with sucrose, cellulose derivatives, or other suitable coating agents.

Tablets can be prepared in a variety of ways such as direct compression, dry or wet granulation, or hot doping.

A pharmaceutical composition in the form of a gelatin capsule can be obtained by mixing the active principle with a solvent and filling the resulting mixture into soft or hard capsules.

For parenteral administration, aqueous suspensions, isotonic saline solutions or sterile injectable solutions are used, the compatible agents of which contain pharmacological agents such as propylene glycol or butylene glycol.

Although the invention has been described with reference to the disclosed embodiments, it should be apparent to those skilled in the art that the specific experiments described in detail are for the purpose of illustrating the present invention only and should not be construed as limiting the scope of the invention in any way. It should be clear that it is possible to carry out various modifications without departing from the essence of the present invention.

### Therapeutic Methods

The compounds of the present invention are inhibitors of CYP11 B2 (aldosterone synthase), and therefore they are useful agents for the therapy and/or prevention of diseases or conditions that can be alleviated by reducing the level of aldosterone. Due to their ability to inhibit aldosterone synthase, the compounds of the present invention are useful in the treatment and/or reduction of the risk of developing: primary hyperaldosteronism (Conn's syndrome), chronic heart failure, chronic heart failure with preserved ejection fraction, left ventricular dysfunction, left ventricular hypertrophy, arterial hypertension, resistant arterial hypertension, pulmonary arterial hypertension, coronary heart disease, liver cirrhosis, metabolic syndrome, chronic kidney disease, glomerulosclerosis, glomerulonephritis, nephritic syndrome, focal segmental glomerulosclerosis, diabetic nephropathy, remodeling of the heart and blood vessels, postinfarction cardiosclerosis, atherosclerosis, increased collagen formation, endothelial dysfunction, hypokalemia and insulin resistance.

These disorders are well characterized in humans, but are also present in other mammals with a similar etiology and can be treated with the pharmaceutical compositions of the present invention.

For therapeutic use, the compounds of the invention may be administered by pharmaceutical composition in any pharmaceutical dosage form by any route of administration. Dosage forms generally include a pharmaceutically acceptable carrier suitable for the particular dosage form chosen. In particular, the compound of the invention may be administered daily for a period of time necessary for the treatment and/or prevention of diseases relevant to the patient, including a course of therapy lasting days, months, years or the entire life of the patient. Routes of administration include, but are not limited to, intravenous, intramuscular, oral, subcutaneous, intraocular, inhalation, intranasal, and sublingual. Preferred routes of administration are oral and intravenous.

The invention also relates to a pharmaceutical composition containing a daily dose of said compound in the form of a fixed dosage unit, and to a combination containing said pharmaceutical composition or said compound. In a preferred embodiment, said composition for use according to the invention is administered once a day at a dosage of 1 mg or more of the selected compound of the invention. The preferred dosage is 1-500 mg. The most preferred dosage is 10-200 mg.

One or more additional pharmacologically active agents may be administered in combination with a compound of formula I, Ia, IIa, Ib, IIb, Ic, Id, IId, Ie, If, IIId. Generally, any additional single or multiple active agents other than a compound of formula I, Ia, IIa, Ib, IIb, Ic, Id, IId, Ie, If, IIId including but not limited to antihypertensive agents, antidiabetic agents, and/or antiobesity agents, may be used in any combination with a compound of formula I, Ia, IIa, Ib, IIb, Ic, Id, IId, Ie, If, IIId in a single or separate dosage form allowing simultaneous or sequential therapeutic action of the active agents.

1. Compound of the general formula (I) as defined hereinafter: 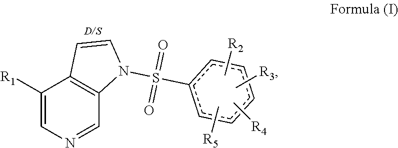
or a pharmaceutically acceptable salt, solvate or hydrate thereof, wherein:
R₁ is a 5-6 membered heteroaryl containing 0-1 oxygen, 0-1 sulfur and/or 0-4 nitrogen, optionally substituted with 1-5 substituents R1a,
R1a is independently selected and represent hydrogen, deuterium, halogen, (C1-C3) alkyl, (C3-C5) cycloalkyl, —C(═O)—CH₃, partially or fully halogenated (C1-C3) alkyl, (C1-C3) alkylsulfonyl, cyano, N,N-dimethylamine, N-methylamine, —C(═O)—NH—(C1-C5)-alkyl;
R₂, R₃, R₄, R₅ are each independently selected and are:
(i) hydrogen, deuterium, halogen, (C1-C3)-alkyl, (C3-C5)-cycloalkyl, —C(═O)—CH₃, partially or fully halogenated (C1-C3)alkyl, or
(ii) —OH, —SH, or
(iii) —(C1-C3)-alkoxy, 3-7-membered heterocyclyl containing 0-2 nitrogen atoms, 0-1 oxygen atom and/or 0-1 sulfur atom, (C3-C7)-cycloalkyl, —S—(C1-C3)-alkyl, —(C3-C7)-cycloalkyl-S—(C1-C3)-alkyl, wherein each of the above groups is optionally partially or completely substituted with substituents selected from —OH, deuterium, 1-4 halogen atoms, (C1-C3)-alkoxy, (C3-C7)-cycloalkyl, oxo group, —(C1-C3)-alkyl-(C3-C6)-cycloalkyl, (C1-C5)-alkyl, partially or fully halogenated (C1-C5) alkyl, (C2-C3) alkenyl, (C2-C3) alkynyl, (C1-C3) alkoxy, —NH₂, 3-7-membered heterocycle containing one oxygen atom, (C1-C3)-alkylamino, di(C1-C3)-alkylamino or 3-7-membered heterocycle containing one nitrogen atom and optionally 1-2 oxygen and/or sulfur heteroatoms; or
(iv) 4-7-membered heterocyclyl containing 1-2 nitrogen atoms and 0-1 oxygen atom and/or 0-1 sulfur atom, necessarily substituted with 1-4 substituents selected from oxo group or —(C1-C3)-alkyl-(C3-C6)-cycloalkyl, and also optionally substituted with 1-3 substituents selected from deuterium, halogen, (C1-C5)-alkyl, partially or fully halogenated (C1-C5) alkyl, (C2-C3) alkenyl, (C2-C3) alkynyl and (C1-C3) alkoxy, or
(v) —NRaRb, where each Ra and Rb:
is independently hydrogen, deuterium, (C3-C7)-cycloalkyl, —(C1-C3)-alkyl-N(CH₃)₂, (C1-C3)-alkyl, partially or fully halogenated (C1-C3)-alkyl, 3-7 membered heterocyclyl containing 0-1 oxygen atom, 0-1 sulfur atom and/or 0-4 nitrogen atoms, optionally substituted with 1-2 substituents selected from (C1-C3)-alkyl or partially or fully halogenated (C1-C3)-alkyl,
5-7-membered heteroaryl containing 0-1 oxygen atom, 0-1 sulfur atom and/or 0-4 nitrogen atoms, said heteroaryl being optionally substituted with 1-2 substituents selected from deuterium, halogen, (C1-C3)-alkyl or partially or fully halogenated (C1-C3)-alkyl,
alternatively, Ra and Rb together with the nitrogen atom to which they are attached form a 4-7 membered heterocyclyl containing 1-2 nitrogen atoms and 0-1 oxygen atom and/or 0-1 sulfur atom, optionally substituted with 1-4 substituents selected from (C1-C5)-alkyl, partially or fully halogenated (C1-C5)-alkyl, oxo group, —(C1-C3)-alkyl-(C3-C6)-cycloalkyl, (C2-C3)-alkenyl, (C2-C3)-alkynyl, (C1-C3) alkoxy or
alternatively, Ra and Rb together with the nitrogen atom to which they are attached form a 6-14-membered bicyclic heterocyclyl containing 1-2 nitrogen atoms and 0-1 oxygen and/or 0-1 sulfur atoms, optionally substituted with 1-4 substituents selected from deuterium, halogen, (C1-C5)-alkyl, partially or fully halogenated (C1-C5)-alkyl, (C2-C3)-alkenyl, (C2-C3) alkynyl, oxo group or —(C1-C3)-alkyl-(C3-C6)-cycloalkyl or (C1-C3) alkoxy;
wherein at least two of R₂, R₃, R₄, R₅ is other than hydrogen;
D/S—means the presence of a double or single bond;
dashed bonds mean the rings are aromatic.

2. A compound according to claim 1, wherein at least three of R₂, R₃, R₄ and R₅ are other than hydrogen.

3. A compound according to claim 1, wherein R₂, R₃, R₄H R₅ are each independently selected and represent:
(i) hydrogen, deuterium, halogen, (C1-C3)-alkyl, (C3-C5)-cycloalkyl, —C(═O)—CH₃, partially or fully halogenated (C1-C3)-alkyl;
(ii) a group selected from (C1-C3)-alkoxy, (C3-C7)cycloalkyl, halogenated (C1-C3)alkoxy group containing 1-4 halogen atoms;
(iii) —NRaRb, where each Ra and Rb:
is independently hydrogen, deuterium, (C3-C7)-cycloalkyl, —(C1-C3)-alkyl-N(CH₃)₂, (C1-C3)-alkyl, partially or fully halogenated (C1-C3)-alkyl, 3-7 membered heterocyclyl containing 0-1 oxygen atom, 0-1 sulfur atom and/or 0-4 nitrogen atoms, optionally substituted with 1-2 substituents selected from (C1-C3)-alkyl, partially or fully halogenated (C1-C3)-alkyl, 5-7 membered heteroaryl containing 0-1 oxygen atom, 0-1 sulfur atom and/or 0-4 nitrogen atoms, optionally substituted with 1-2 substituents selected from (C1-C3)-alkyl, partially or fully halogenated (C1-C3)-alkyl,
alternatively, Ra and Rb together with the nitrogen atom to which they are attached form a 4-7 membered heterocyclyl containing 1-2 nitrogen atoms and 0-1 oxygen atom and/or 0-1 sulfur atom, optionally substituted with 1-2 substituents selected from (C1-C3)-alkyl, partially or fully halogenated (C1-C3)-alkyl, or
alternatively, Ra and Rb together with the nitrogen atom to which they are attached form a 6-14-membered bicyclic heterocyclyl containing 1-2 nitrogen atoms and 0-1 oxygen and/or 0-1 sulfur atom, optionally substituted with 1-4 substituents selected from deuterium, halogen, (C1-C3)-alkyl, partially or fully halogenated (C1-C3)-alkyl, (C2-C3)-alkenyl, (C2-C3)-alkynyl or (C1-C3)-alkoxy;
wherein at least two of R₂, R₃, R₄, R₅ is other than hydrogen.

4. A compound according to claim 1, wherein at least one of the radicals R₂, R₃, R₄, R₅ is in the para position and each is independently selected and represent:
—OH, —SH, (C1-C3)-alkoxy, 3-7-membered heterocyclyl containing 0-2 nitrogen atom, 0-1 oxygen atom and/or 0-1 sulfur atom, (C3-C7)-cycloalkyl, —S—(C1-C3)-alkyl, (C3-C7)-cycloalkyl-S—(C1-C3)-alkyl, wherein each of the above groups is optionally partially or completely substituted with substituents selected from —OH, 1-4 halogen atoms, (C1-C3)-alkoxy, (C3-C7)-cycloalkyl, 3-7-membered heterocycle containing one oxygen atom, —NH₂, (C1-C3)-alkylamino, di(C1-C3)-alkylamino or 3-7-membered heterocycle containing one nitrogen atom and optionally 1-2 oxygen and sulfur heteroatoms; or
NRaRb, where each Ra and Rb:
is independently hydrogen, (C3-C7)-cycloalkyl, —(C1-C3)-alkyl-N(CH₃)₂, (C1-C3)-alkyl, partially or fully halogenated (C1-C3)-alkyl, 3-7 membered heterocycle containing 0-1 oxygen atom, 0-1 sulfur and 1-4 nitrogen atoms, optionally substituted with 1-2 substituents selected from (C1-C3)-alkyl or partially or fully halogenated (C1-C3)-alkyl, 5-7 membered heteroaryl containing 1-2 nitrogen atom, wherein said heteroaryl is optionally substituted with 1-2 substituents selected from (C1-C3)-alkyl or partially or fully halogenated (C1-C3)-alkyl,
alternatively, Ra and Rb together with the nitrogen atom to which they are attached form a 4-7 membered heterocyclyl containing 1-2 nitrogen atoms and 0-1 oxygen atom and/or 0-1 sulfur atom, optionally substituted with 1-2 substituents selected from (C1-C3)-alkyl, partially or fully halogenated (C1-C3)-alkyl, or
alternatively, Ra and Rb, together with the nitrogen atom to which they are attached, form a 6-14-membered bicyclic heterocyclyl containing 1-2 nitrogen atoms and 0-1 oxygen and/or 0-1 sulfur atoms, optionally substituted with 1-4 substituents selected from halogen, (C1-C3)-alkyl, partially or fully halogenated (C1-C3)-alkyl, (C2-C3)-alkenyl, (C2-C3)-alkynyl or (C1-C3) alkoxy.

5. A compound according to claim 1, wherein R₁ is independently selected from pyridinyl, pyrazinyl, pyrimidinyl, pyridazinyl, oxazyl, wherein R₁ is optionally substituted with a group selected from methyl, methylsulfonyl, halogen, methylamino, cyano and cyclopropyl.

6. A compound according to claim 1, wherein two of R₂, R₃, R₄, R₅ are in meta position and are a group selected from methyl or halogen, one of R₂, R₃, R₄, R₅ is hydrogen and one of R₂, R₃, R₄, R₅ is in para position and is a group selected from methoxy, isopropyloxy, cyclopropylmethoxy, difluoroethoxy, —NH₂, —N(CH₃)₂, cyclopropylamino, —N(CH₃)-cyclobutyl, —N(CH₃)-methylthiazolyl, methylpiperidylamino, —NH—(C1-C3)-alkyl-N(CH₃)₂, morpholinyl, methylpiperazinyl, optionally methyl-substituted azetidinyl, thiomorpholinyl, optionally methyl-substituted hexahydropyrrolopyrrolyl, diazaspirononanyl, optionally substituted with methyl octahydropyrrolopyridinyl.

7. A compound according to claim 1, wherein the compound of formula (I) is a compound whose structure is described by Formula (Ia): 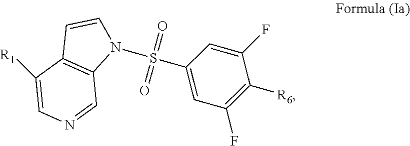
or a pharmaceutically acceptable salt or solvate or hydrate thereof, wherein:
R₁ as defined in claim 1,
R₆ is chosen independently and is:
—OH, —SH, —(C1-C3)-alkoxy, 3-7 membered heterocyclyl containing 0-1 oxygen atoms and/or 0-1 sulfur atom, (C3-C7)-cycloalkyl, —S—(C1-C3)-alkyl, (C3-C7)-cycloalkyl-S—(C1-C3)-alkyl, wherein each of the above groups is optionally partially or completely substituted with substituents selected from —OH, 1-4 halogen atoms, (C1-C3)-alkoxy, (C3-C7)-cycloalkyl, 3-7 membered heterocycle containing one oxygen atom, —NH₂, (C1-C3)-alkylamino, di(C1-C3)-alkylamino or 3-7-membered heterocycle containing one nitrogen atom and optionally 1-2 heteroatoms selected from oxygen and sulfur, or
—NRaRb, where each Ra and Rb:
is independently hydrogen, (C3-C7)-cycloalkyl, —(C1-C3)-alkyl-N(CH₃)₂, (C1-C3)-alkyl, partially or fully halogenated (C1-C3)-alkyl, 3-7 membered heterocycle containing 0-1 oxygen atom, 0-1 sulfur and/or 1-4 nitrogen, optionally substituted with 1-2 substituents selected from (C1-C3)-alkyl or partially or fully halogenated (C1-C3)-alkyl, 5-7 membered heteroaryl containing 0-1 oxygen, 0-1 sulfur and/or 1-4 nitrogen, said heteroaryl being optionally substituted with 1-2 substituents selected from (C1-C3)-alkyl or partially or fully halogenated (C1-C3)-alkyl,
alternatively Ra and Rb together with the nitrogen atom to which they are attached form a 4-7 membered heterocyclyl containing 1-2 nitrogen atoms and 0-1 oxygen atoms and/or 0-1 sulfur atoms, optionally substituted with 1-2 substituents selected from (C1-C3)-alkyl, partially or fully halogenated (C1-C3)-alkyl, or
alternatively, Ra and Rb, together with the nitrogen atom to which they are attached, form a 6-14-membered bicyclic heterocyclyl containing 1-2 nitrogen atoms and 0-1 oxygen and/or 0-1 sulfur atoms, optionally substituted with 1-4 substituents selected from halogen, (C1-C3)-alkyl, partially or fully halogenated (C1-C3)-alkyl, (C2-C3)-alkenyl, (C2-C3) alkynyl or (C1-C3) alkoxy.

8. A compound according to claim 1, wherein the compound of formula (I) is a compound whose structure is described by Formula (IIa): 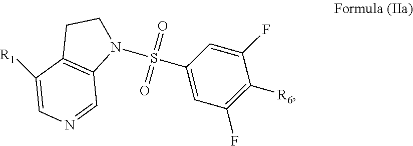
or a pharmaceutically acceptable salt, solvate or hydrate thereof, wherein:
R₁ and R₆ as defined in claim 7.

9. A compound according to claim 1, wherein the compound of formula (I) is a compound whose structure is described by Formula (Ib): 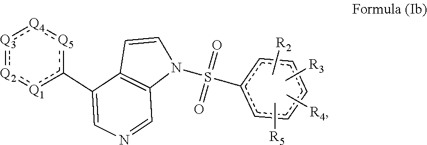
or a pharmaceutically acceptable salt, solvate or hydrate thereof, wherein:
Q₁, Q₂, Q₃, Q₄, Q₅ are each independently selected and are nitrogen or carbon, where no more than two of Q₁, Q₂, Q₃, Q₄, and Q₅ are nitrogen, and said carbon is optionally substituted with 1-5 substituents R1a,
R₂, R₃, R₄, R₅ and R1a as defined in claim 1;
dotted bonds mean the rings are aromatic.

10. A compound according to claim 1, wherein the compound of formula (I) is a compound whose structure is described by Formula (Ic): 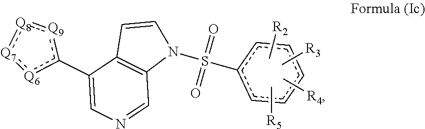
or a pharmaceutically acceptable salt, solvate or hydrate thereof, wherein:
Q₆, Q₇, Q₈, Q₉ are each independently selected and are oxygen, carbon, or nitrogen, with no more than two of Q₆, Q₇, Q₈ and Q₉ being nitrogen or oxygen, and said carbon is optionally substituted with 1-2 R1a groups, R₂, R₃, R₄, R₅ and R1a as defined in claim 1,
dotted bonds mean the rings are aromatic.

11. A compound according to claim 1, wherein the compound of formula (I) is a compound whose structure is described by Formula (Id): 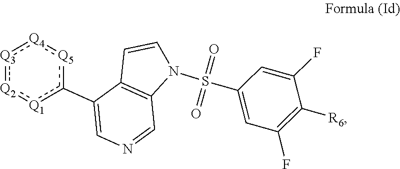
or a pharmaceutically acceptable salt, solvate or hydrate thereof, wherein:
Q₁, Q₂, Q₃, Q₄, Q₅ as defined in claim 9;
R₆ as defined in claim 7.

12. A compound according to claim 1, wherein the compound of formula (I) is a compound whose structure is described by Formula (Ie): 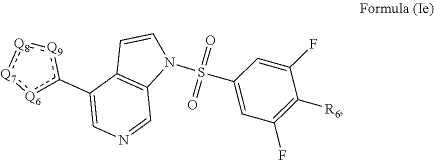
or a pharmaceutically acceptable salt, solvate or hydrate thereof, wherein:
Q₆, Q₇, Q₈, Q₉ as defined in claim 10;
R₆ as defined in claim 7.

13. A compound according to claim 1, wherein the compound of formula (I) is a compound whose structure is described by Formula (If): 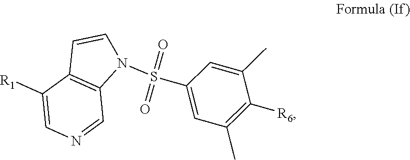
or a pharmaceutically acceptable salt, solvate or hydrate thereof, wherein:
R₁ as defined in claim 1;
R₆ as defined in claim 7.

14. A compound according to claim 1, wherein the compound of formula (I) is a compound whose structure is described by Formula (IIb): 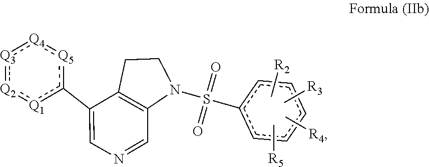
or a pharmaceutically acceptable salt, solvate or hydrate thereof, wherein:
Q₁, Q₂, Q₃, Q₄, Q₅ as defined in claim 9;
R₂, R₃, R₄ and R₅ as defined in claim 1;
dotted bonds mean the rings are aromatic.

15. A compound according to claim 1, wherein the compound of formula (I) is a compound whose structure is described by Formula (IId): 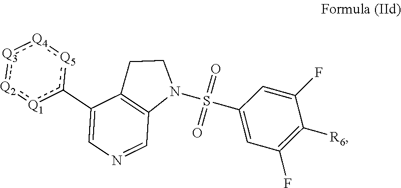
or a pharmaceutically acceptable salt, solvate or hydrate thereof, wherein:
Q₁, Q₂, Q₃, Q₄, Q₅ and as defined in claim 9;
R₆ as defined in claim 7.

16. A compound according to claim 1, wherein the compound of formula (I) is a compound whose structure is described by Formula (IIId): 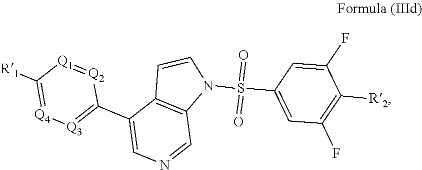
or a pharmaceutically acceptable salt, solvate or hydrate thereof, wherein:
Q₁, Q₂, Q₃, Q₄ are each independently represent nitrogen or carbon;
if R′₁ independently represents hydrogen, deuterium, halogen, (C1-C3) alkyl, (C3-C5) cycloalkyl, —C(═O)—CH₃, partially or fully halogenated (C1-C3) alkyl, (C1-C3) alkylsulfonyl, cyano, N,N-dimethylamine, N-methylamine, then R′₂ independently represents 4-7-membered heterocyclyl containing 1-2 nitrogen atoms, 0-1 oxygen atom and/or 0-1 sulfur atom, necessary substituted 1-2 substituents selected from oxo group or —(C1-C3)-alkyl-(C3-C6)-cycloalkyl, and also optionally substituted with 1-3 substituents selected from deuterium, halogen, (C1-C5)-alkyl, partially or fully halogenated (C1-C5) alkyl, (C2-C3) alkenyl, (C2-C3) alkynyl and (C1-C3) alkoxy; or
if R′₁ independently represents —C(═O)—NH—(C1-C5)-alkyl; then R₂ independently represents:
NRcRd, where each Rc and Rd:
is independently hydrogen, deuterium, (C3-C7)-cycloalkyl, —(C1-C3)-alkyl-N(CH₃)₂, (C1-C3)-alkyl, partially or fully halogenated (C1-C3)-alkyl, 3-7 membered heterocyclyl containing 0-1 oxygen atom, 0-1 sulfur atom and/or 0-4 nitrogen atoms, optionally substituted with 1-2 substituents selected from (C1-C3)-alkyl or partially or fully halogenated (C1-C3)-alkyl, 5-7-membered heteroaryl containing 0-1 oxygen atom, 0-1 sulfur atom and/or 0-4 nitrogen atoms, said heteroaryl being optionally substituted with 1-2 substituents selected from (C1-C3)-alkyl or partially or fully halogenated (C1-C3)-alkyl,
alternatively, Rc and Rd together with the nitrogen atom to which they are attached form a 4-7 membered heterocyclyl containing 1-2 nitrogen atoms and 0-1 oxygen atom and/or 0-1 sulfur atom, optionally substituted with 1-4 substituents selected from deuterium, halogen, (C1-C5)-alkyl, partially or fully halogenated (C1-C5)-alkyl, (C2-C3)-alkenyl, oxo group, —(C1-C3)-alkyl-(C3-C6)-cycloalkyl or (C1-C3) alkoxy, or
alternatively, Rc and Rd together with the nitrogen atom to which they are attached form a 6-14-membered bicyclic heterocyclyl containing 1-2 nitrogen atoms and 0-1 oxygen and/or 0-1 sulfur atoms, optionally substituted with 1-4 substituents selected from deuterium, halogen, (C1-C5)-alkyl, partially or fully halogenated (C1-C5)-alkyl, (C2-C3)-alkenyl, (C2-C3) alkynyl, oxo group, —(C1-C3)-alkyl-(C3-C6)-cycloalkyl or (C1-C3) alkoxy.

17. The compound according to claim 1, selected from the group:
1-(4-methoxy-3,5-dimethylphenyl)sulfonyl-4-(5-methyl-2-pyridyl)pyrrolo[2,3-c]pyridine;
1-((4-Methoxy-3,5-dimethylphenyl)sulfonyl)-4-(5-methylpyrazin-2-yl)-1H-pyrrolo[2,3-c]pyridine;
1-((4-methoxy-3,5-dimethylphenyl)sulfonyl)-4-(5-methylpyrimidin-2-yl)-1H-pyrrolo[2,3-c]pyridine;
1-(4-methoxy-3,5-dimethylphenyl)sulfonyl-4-(6-methylpyridazin-3-yl)pyrrolo[2,3-c]pyridine;
1-(4-methoxy-3,5-dimethylphenyl)sulfonyl-4-(5-methylsulfonyl-2-pyridyl)pyrrolo[2,3-c]pyridine;
1-((3,5-difluoro-4-isopropoxyphenyl)sulfonyl)-4-(pyridin-2-yl)-1H-pyrrolo[2,3-c]pyridine;
1-((4-(cyclopropylmethoxy)-3,5-difluorophenyl)sulfonyl)-4-(pyridin-2-yl)-1H-pyrrolo[2,3-c]pyridine;
1-((4-(2,2-difluoroethoxy)-3,5-difluorophenyl)sulfonyl)-4-(pyridin-2-yl)-1H-pyrrolo[2,3-c]pyridine;
2,6-difluoro-N,N-dimethyl-4-((4-(pyridin-2-yl)-1H-pyrrolo[2,3-c]pyridin-1-yl)sulfonyl)aniline;
4-(2,6-difluoro-4-((4-(pyridin-2-yl)-1H-pyrrolo[2,3-c]pyridin-1-yl)sulfonyl)phenyl)morpholine;
2,6-difluoro-4-((4-(pyridin-2-yl)-1H-pyrrolo[2,3-c]pyridin-1-yl)sulfonyl)aniline;
1-((3,5-difluoro-4-(4-methylpiperazin-1-yl)phenyl)sulfonyl)-4-(pyridin-2-yl)-1H-pyrrolo[2,3-c]pyridine;
2,6-difluoro-4-((4-(pyridin-2-yl)-1H-pyrrolo[2,3-c]pyridin-1-yl)sulfonyl)aniline;
N-cyclobutyl-2,6-difluoro-N-methyl-4-((4-(pyridin-2-yl)-1H-pyrrolo[2,3-c]pyridin-1-yl)sulfonyl)aniline;
2,6-difluoro-N,N-dimethyl-4-((4-(pyridin-2-yl)-2,3-dihydro-1H-pyrrolo[2,3-c]pyridin-1-yl)sulfonyl)aniline;
2,6-difluoro-4-((4-(pyridin-2-yl)-2,3-dehydro-H-pyrrolo[2,3-c]pyridin-1-yl)sulfonyl)phenol;
1-((3,5-difluoro-4-(4-methylpiperazin-1-yl)phenyl)sulfonyl)-4-(pyridin-2-yl)-2,3-dihydro-1H-pyrrolo[2,3-c]pyridine;
1-((3,5-difluoro-4-methoxyphenyl)sulfonyl)-4-(pyridin-2-yl)-1H-pyrrolo[2,3-c]pyridine;
1-((3,5-difluoro-4-(3-methylazetidin-1-yl)phenyl)sulfonyl)-4-(pyridin-2-yl)-1H-pyrrolo[2,3-c]pyridine;
N-(2,6-difluoro-4-((4-(pyridin-2-yl)-1H-pyrrolo[2,3-c]pyridin-1-yl)sulfonyl)phenyl)-N,4-dimethylthiazol-2-amine;
N-(2,6-difluoro-4-((4-(pyridin-2-yl)-1H-pyrrolo[2,3-c]pyridin-1-yl)sulfonyl)phenyl)-1-methylpiperidin-4-amine;
N1-(2,6-difluoro-4-((4-(pyridin-2-yl)-1H-pyrrolo[2,3-c]pyridin-1-yl)sulfonyl)phenyl)-N2,N2-dimethylethane-1,2-diamine;
4-(2,6-difluoro-4-((4-(pyridin-2-yl)-1H-pyrrolo[2,3-c]pyridin-1-yl)sulfonyl)phenyl)thiomorpholine;
1-((3,5-difluoro-4-(5-methylhexahydropyrrolo[3,4-c]pyrrol-2(1H)-yl)phenyl)sulfonyl)-4-(pyridin-2-yl)-1H-pyrrolo [2,3-s]pyridine;
1-((3,5-difluoro-4-(5-methyloctahydro-2H-pyrrolo[3,4-c]pyridin-2-yl)phenyl)sulfonyl)-4-(pyridin-2-yl)-1H-pyrrolo[2,3-c]pyridine;
2-(2,6-difluoro-4-((4-(pyridin-2-yl)-1H-pyrrolo[2,3-c]pyridin-1-yl)sulfonyl)phenyl)-2,7-diazaspiro[4.4]nonane;
2-(1-((3,5-difluoro-4-(4-methylpiperazin-1-yl)phenyl)sulfonyl)-1H-pyrrolo[2,3-c]pyridin-4-yl)oxazol-4-carbonitrile;
5-(1-((3,5-difluoro-4-(4-methylpiperazin-1-yl)phenyl)sulfonyl)-1H-pyrrolo[2,3-c]pyridin-4-yl)picolinonitrile;
1-((3,5-difluoro-4-(4-methylpiperazin-1-yl)phenyl)sulfonyl)-4-(5-fluoropyrimidin-2-yl)-1H-pyrrolo[2,3-c]pyridine;
5-(1-((3,5-difluoro-4-(4-methylpiperazin-1-yl)phenyl)sulfonyl)-1H-pyrrolo[2,3-c]pyridin-4-yl)-N-methylpyrazin-2-amine;
4-(5-cyclopropylpyridin-2-yl)-1-((3,5-difluoro-4-(4-methylpiperazin-1-yl)phenyl)sulfonyl)-1H-pyrrolo[2,3-c]pyridine;
2,6-difluoro-4-((4-(5-methylpyrazin-2-yl)-1H-pyrrolo[2,3-c]pyridin-1-yl)sulfonyl)aniline;
2,6-difluoro-N,N-dimethyl-4-((4-(5-methylpyridin-2-yl)-1H-pyrrolo[2,3-c]pyridin-1-yl)sulfonyl)aniline;
4-(2,6-difluoro-4-((4-(pyridin-2-yl)-1H-pyrrolo[2,3-c]pyridin-1-yl)sulfonyl)phenyl)-1-methylpiperazin-2-one;
1-((4-(4-(cyclopropylmethyl)piperazin-1-yl)-3,5-difluorophenyl)sulfonyl)-4-(pyridin-2-yl)-1H-pyrrolo[2,3-c]pyridine;
N-(tert-butyl)-5-(1-((3,5-difluoro-4-(5-methylhexahydropyrrolo[3,4-c]pyrrol-2(1H)-yl)phenyl)sulfonyl)-1H-pyrrolo[2,3-c]pyridin-4-yl)picolinamide;
1-((3,5-difluoro-4-(piperazin-1-yl)phenyl)sulfonyl)-4-(pyridin-2-yl)-1H-pyrrolo[2,3-c]pyridine;
6-(1-((3,5-difluoro-4-(4-methylpiperazin-1-yl)phenyl)sulfonyl)-1H-pyrrolo[2,3-c]pyridin-4-yl)-N-methylpyridin-3-amine;
5-(1-((3,5-difluoro-4-(4-methylpiperazin-1-yl)phenyl)sulfonyl)-1H-pyrrolo[2,3-c]pyridin-4-yl)-N-methylpyridin-2-amine;
6-(1-((3,5-difluoro-4-(4-methylpiperazin-1-yl)phenyl)sulfonyl)-1H-pyrrolo[2,3-c]pyridin-4-yl)-N,N-dimethylpyridin-3-amine;
5-(1-((3,5-difluoro-4-(4-methylpiperazin-1-yl)phenyl)sulfonyl)-1H-pyrrolo[2,3-c]pyridin-4-yl)pyrazine-2-carbonitrile;
6-(1-((3,5-difluoro-4-(4-methylpiperazin-1-yl)phenyl)sulfonyl)-1H-pyrrolo[2,3-c]pyridin-4-yl)nicotinonitrile;
6-(1-((3,5-difluoro-4-(4-methylpiperazin-1-yl)phenyl)sulfonyl)-1H-pyrrolo[2,3-c]pyridin-4-yl)-N-methylpyridazin-3-amine;
2-(1-((3,5-difluoro-4-(4-methylpiperazin-1-yl)phenyl)sulfonyl)-1H-pyrrolo [2,3-c]pyridin-4-yl)-N-methylpyrimidin-5-amine;
5-(1-((3,5-difluoro-4-(4-methylpiperazin-1-yl)phenyl)sulfonyl)-1H-pyrrolo [2,3-c]pyridin-4-yl)pyrimidine-2-carbonitrile;
6-(1-((3,5-difluoro-4-(4-methylpiperazin-1-yl)phenyl)sulfonyl)-1H-pyrrolo[2,3-c]pyridin-4-yl)pyridazine-3-carbonitrile;
1-((3,5-difluoro-4-(4-isopropylpiperazin-1-yl)phenyl)sulfonyl)-4-(pyridin-2-yl)-1H-pyrrolo[2,3-c]pyridine;
1-((4-(4-cyclopropylpiperazin-1-yl)-3,5-difluorophenyl)sulfonyl)-4-(pyridin-2-yl)-1H-pyrrolo[2,3-c]pyridine;
1-((3,5-difluoro-4-(4-methyl-1,4-diazepan-1-yl)phenyl)sulfonyl)-4-(pyridin-2-yl)-1H-pyrrolo[2,3-c]pyridine;
1-((4-(1,4-diazepan-1-yl)-3,5-difluorophenyl)sulfonyl)-4-(pyridin-2-yl)-1H-pyrrolo[2,3-c]pyridine;
1-(2,6-difluoro-4-((4-(pyridin-2-yl)-1H-pyrrolo[2,3-c]pyridin-1-yl)sulfonyl)phenyl)piperidin-4-amine;
1-((3,5-difluoro-4-(6-methyl-2,6-diazaspiro[3.3]heptan-2-yl)phenyl)sulfonyl)-4-(pyridin-2-yl)-1H-pyrrolo[2,3-c]pyridine;
1-((3,5-difluoro-4-(5-methyl-2,5-diazabicyclo[2.2.1]heptan-2-yl)phenyl)sulfonyl)-4-(pyridin-2-yl)-1H-pyrrolo[2,3-c]pyridine;
6-(2,6-difluoro-4-((4-(pyridin-2-yl)-1H-pyrrolo[2,3-c]pyridin-1-yl)sulfonyl)phenyl)-3-methyl-3,6-diazabicyclo[3.1.1]heptane;
1-((3,5-difluoro-4-(3-methyl-3,8-diazabicyclo[3.2.1]octan-8-yl)phenyl)sulfonyl)-4-(pyridin-2-yl)-1H-pyrrolo[2,3-c]pyridine;
1-((4-(1,4-diazabicyclo[3.2.1]octan-4-yl)-3,5-difluorophenyl)sulfonyl)-4-(pyridin-2-yl)-1H-pyrrolo[2,3-c]pyridine;
1-((3,5-difluoro-4-(2,6-diazaspiro[3.3]heptan-2-yl)phenyl)sulfonyl)-4-(pyridin-2-yl)-1H-pyrrolo[2,3-c]pyridine;
5-(1-((3,5-difluoro-4-(5-methylhexahydropyrrolo[3,4-c]pyrrol-2(1H)-yl)phenyl)sulfonyl)-1H-pyrrolo[2,3-c]pyridin-4-yl)-N-methylpyrazin-2-amine;
5-(1-((3,5-difluoro-4-(5-methylhexahydropyrrolo[3,4-c]pyrrol-2(1H)-yl)phenyl)sulfonyl)-1H-pyrrolo[2,3-c]pyridin-4-yl)pyrazine-2-carbonitrile;
5-(1-((3,5-difluoro-4-(5-methylhexahydropyrrolo[3,4-c]pyrrol-2(1H)-yl)phenyl)sulfonyl)-1H-pyrrolo[2,3-c]pyridin-4-yl)picolinonitrile;
1-((4-(2,5-diazabicyclo[2.2.1]heptan-2-yl)-3,5-difluorophenyl)sulfonyl)-4-(pyridin-2-yl)-1H-pyrrolo[2,3-c]pyridine (HCl salt);
1-((4-(3,8-diazabicyclo[3.2.1]octan-8-yl)-3,5-difluorophenyl)sulfonyl)-4-(pyridin-2-yl)-1H-pyrrolo[2,3-c]pyridine;
1-((3,5-difluoro-4-(piperazin-1-yl)phenyl)sulfonyl)-4-(pyridin-2-yl)-1H-pyrrolo[2,3-c]pyridine (HCl salt);
5-(1-((3,5-difluoro-4-(hexahydropyrrolo[3,4-c]pyrrol-2(1H)-yl)phenyl)sulfonyl)-1H-pyrrolo[2,3-c]pyridin-4-yl)picolinonitrile;
1-((3,5-difluoro-4-(4-methylpiperazin-1-yl)phenyl)sulfonyl)-4-(pyridin-2-yl)-1H-pyrrolo[2,3-c]pyridine (HCl salt).

18. A use of a compound according to claim 1 for inhibiting of CYP11B2.

19. A use of a compound according to claim 1 in lowering aldosterone level in a subject.

20. The use according to claim 19, wherein the subject is a human.

21. A pharmaceutical composition for the treatment and/or prevention of a disease or condition that is mediated or maintained by CYP11B2 activity, comprising a therapeutically or prophylactically effective amount of a compound according to claim 1 and at least one pharmaceutically acceptable excipient.

22. The pharmaceutical composition according to claim 21, characterized in that the pharmaceutically acceptable excipient is a carrier, adjuvant and/or solvent.

23. The pharmaceutical composition according to claim 21, characterized in that the disease is selected from the group including primary hyperaldosteronism, chronic heart failure, chronic heart failure with preserved ejection fraction, left ventricular dysfunction, left ventricular hypertrophy, arterial hypertension, resistant arterial hypertension, pulmonary arterial hypertension, coronary heart disease, liver cirrhosis, metabolic syndrome, chronic kidney disease, glomerulosclerosis, glomerulonephritis, nephritic syndrome, focal segmental glomerulosclerosis, diabetic nephropathy, cardiac and vascular remodeling, postinfarction cardiosclerosis, atherosclerosis, increased collagen formation, endothelial dysfunction, hypokalemia, or insulin resistance.

24. The pharmaceutical composition according to claim 23, characterized in that primary hyperaldosteronism is Conn's syndrome, including the following diseases or conditions: aldosterone-producing adenoma, idiopathic hyperaldosteronism, unilateral or bilateral adrenal hyperplasia, familial hyperaldosteronism of the first and second type, aldosterone-producing carcinoma and aldosterone ectopic.

25. A method of treating and/or preventing a disease or condition that is mediated or maintained by CYP11B2 activity in a subject, comprising administering a therapeutically or prophylactically effective amount of a compound according to claim 1.

26. The method according to claim 25, characterized in that the disease is selected from the group including primary hyperaldosteronism, chronic heart failure, chronic heart failure with preserved ejection fraction, left ventricular dysfunction, left ventricular hypertrophy, arterial hypertension, resistant arterial hypertension, pulmonary arterial hypertension, coronary heart disease, liver cirrhosis, metabolic syndrome, chronic kidney disease, glomerulosclerosis, glomerulonephritis, nephritic syndrome, focal segmental glomerulosclerosis, diabetic nephropathy, cardiac and vascular remodeling, postinfarction cardiosclerosis, atherosclerosis, increased collagen formation, endothelial dysfunction, hypokalemia, or insulin resistance.

27. The method according to claim 25, characterized in that the primary hyperaldosteronism is Conn's syndrome, including the following diseases or conditions: aldosterone-producing adenoma, idiopathic hyperaldosteronism, unilateral or bilateral adrenal hyperplasia, familial hyperaldosteronism of the first and second type, aldosterone-producing carcinoma and aldosteronectopic.

28. The method of claim 23, wherein the subject is a human.
